SEQUENCE LISTING (57 sequences)

SEQ ID NO: 1 - protein, 21 aa, Artificial Sequence
  amino acid sequnces of human TMEM59 transmembrane region
GWILTTTLVLSVMVLLWICCA

SEQ ID NO: 2 - protein, 50 aa, Artificial Sequence
  amino aicd sequence of human TMEM59 hinge region
PHLEQEPTNLRESSLSKMSSDLQMRNSQAHRNFLEDGESDGFLRCLSLNS

SEQ ID NO: 3 - protein, 324 aa, Artificial Sequence
  amino acid sequence of human TMEM59 protein and/or its functional fragments
MAAPKGSLWVRTQLGLPPLLLLTMALAGGSGTASAEAFDSVLGDTASCHRACQLTYPLHTYPKEEELYACQRGCRLFSICQFVDDGIDLNRTKLECESACTEAYSQSDEQYACHLGCQNQLPFAELRQEQLMSLMPKMHLLFPLTLVRSFWSDMMDSAQSFITSSWTFYLQADDGKIVIFQSKPEIQYAPHLEQEPTNLRESSLSKMSSDLQMRNSQAHRNFLEDGESDGFLRCLSLNSGWILTTTLVLSVMVLLWICCATVATAVEQYVPSEKLSIYGDLEFMNEQKLNRYPASSLVVVRSKTEDHEEAGPLPTKVNLAHSEI

SEQ ID NO: 4 - DNA, 975 bp, Artificial Sequence
  nucleotide sequence of human TMEM59 protein and/or its functional fragments
atggcggcgccgaaggggagcctctgggtgaggacccaactggggctcccgccgctgctgctgctgaccatggccttggccggaggttcggggaccgcttcggctgaagcatttgactcggtcttgggtgatacggcgtcttgccaccgggcctgtcagttgacctaccccttgcacacctaccctaaggaagaggagttgtacgcatgtcagagaggttgcaggctgttttcaatttgtcagtttgtggatgatggaattgacttaaatcgaactaaattggaatgtgaatctgcatgtacagaagcatattcccaatctgatgagcaatatgcttgccatcttggttgccagaatcagctgccattcgctgaactgagacaagaacaacttatgtccctgatgccaaaaatgcacctactctttcctctaactctggtgaggtcattctggagtgacatgatggactccgcacagagcttcataacctcttcatggactttttatcttcaagccgatgacggaaaaatagttatattccagtctaagccagaaatccagtacgcaccacatttggagcaggagcctacaaatttgagagaatcatctctaagcaaaatgtcctcagatctgcaaatgagaaattcacaagcgcacaggaattttcttgaagatggagaaagtgatggctttttaagatgcctctctcttaactctgggtggattttaactacaactcttgtcctctcggtgatggtattgctttggatttgttgtgcaactgttgctacagctgtggagcagtatgttccctctgagaagctgagtatctatggtgacttggagtttatgaatgaacaaaagctaaacagatatccagcttcttctcttgtggttgttagatctaaaactgaagatcatgaagaagcagggcctctacctacaaaagtgaatcttgctcattctgaaatttaa

SEQ ID NO: 5 - DNA, 63 bp, Artificial Sequence
  nucleotide sequence of human TMEM59 transmembrane region
gggtggattttaactacaactcttgtcctctcggtgatggtattgctttggatttgttgtgca

SEQ ID NO: 6 - DNA, 150 bp, Artificial Sequence
  nucleotide sequence of human TMEM59 hinge region
ccacatttggagcaggagcctacaaatttgagagaatcatctctaagcaaaatgtcctcagatctgcaaatgagaaattcacaagcgcacaggaattttcttgaagatggagaaagtgatggctttttaagatgcctctctcttaactct

SEQ ID NO: 7 - DNA, 2796 bp, Artificial Sequence
  21D4-59TM CAR-IRES-GFP
atggccttaccagtgaccgccttgctcctgccgctggccttgctgctccacgccgccaggccggccatccagttgacccagtctccatcctccctgtctgcatctgtaggagacagagtcaccatcacttgccgggcaagtcagggcattagcagtgctttagcctggtatcagcagaaaccagggaaagctcctaagctcctgatctatgatgcctccagtttggaaagtggggtcccatcaaggttcagcggcagtggatctgggacagatttcactctcaccatcagcagcctgcagcctgaagattttgcaacttattactgtcaacagtttaatagttacccgtacacttttggccaggggaccaagctggagatcaaaggtggcggtggctcgggcggtggtgggtcgggtggcggcggatctgaggtgcagctggtgcagtctggagcagaggtgaaaaagcccggggagtctctgaagatctcctgtaagggttctggatacagctttagcagcagctggatcggctgggtgcgccagatgcccgggaaaggcctggagtggatggggatcatctatcctgatgactctgataccagatacagtccgtccttccaaggccaggtcaccatctcagccgacaagtccatcaggaccgcctacctgcagtggagcagcctgaaggcctcggacaccgccatgtattactgtgcgagacatgttactatgatttggggagttattattgacttctggggccagggaaccctggtcaccgtctcctcaccacatttggagcaggagcctacaaatttgagagaatcatctctaagcaaaatgtcctcagatctgcaaatgagaaattcacaagcgcacaggaattttcttgaagatggagaaagtgatggctttttaagatgcctctctcttaactctgggtggattttaactacaactcttgtcctctcggtgatggtattgctttggatttgttgtgcaaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaaggatcccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataagcttgccacaacccacaatctagaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 8 - DNA, 135 bp, Artificial Sequence
  nucleotide sequence of CD8 hinge region
accacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgat

SEQ ID NO: 9 - DNA, 336 bp, Artificial Sequence
  nucleotide sequence of CD3 zeta signaling domain
agagtgaagttcagcaggagcgcagacgcccccgcgtaccagcagggccagaaccagctctataacgagctcaatctaggacgaagagaggagtacgatgttttggacaaaagaagaggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 10 - DNA, 141 bp, Artificial Sequence
  nucleotide sequence of human 4-1BB costimulatory domain
cgtttctctgttgttaaacggggcagaaagaagctcctgtatatattcaaacaaccatttatgagaccagtacaaactactcaagaggaagatggctgtagctgccgatttccagaagaagaagaaggaggatgtgaactg

SEQ ID NO: 11 - DNA, 735 bp, Artificial Sequence
  humanized CD19 monoclonal antibody single chain variable region H63
caagtgcagctcgtccaaagcggcggaggagtggtgcagcccggaagaagcctcagactcagctgtgctgccagcggagtcagcctccccgactacggagtgagctgggtgagacaagcccccggcaagggactggaatgggtcgccgtgatttggggcagcgagaccacctactacaactccgctctgaagtctagattcacaatctccagagataattccaaaaataccctctatctgcaaatgaactctctgagggccgaggacaccgccgtgtactactgtgctaagcactactactatggcggcagcttcgccatggattactggggacaaggcacactggtgaccgtctcctccggaagcaccagcggaagcggaaagcccggcagcggcgagggctccacaaaaggagacatccagatgacacaatccccttcctctctgagcgcttccgtcggcgacagagtcaccatcacatgcagagccagccaagacatcagcaagtatctgaactggtaccagcagaagcccggcaaggccatcaaactgctgatctaccacacatctagactgcacagcggagtgccctccagattctccggcagcggcagcggcaccgactttacactgaccattagctctctgcagcccgaggactttgctacctactactgtcagcaaggcgccacactgccctatacctttggccccggcacaaaggtggacatcaag

SEQ ID NO: 12 - DNA, 792 bp, Artificial Sequence
  full-humanized CD19 monoclonal antibody single chain variable region 21D4
atggccttaccagtgaccgccttgctcctgccgctggccttgctgctccacgccgccaggccggccatccagttgacccagtctccatcctccctgtctgcatctgtaggagacagagtcaccatcacttgccgggcaagtcagggcattagcagtgctttagcctggtatcagcagaaaccagggaaagctcctaagctcctgatctatgatgcctccagtttggaaagtggggtcccatcaaggttcagcggcagtggatctgggacagatttcactctcaccatcagcagcctgcagcctgaagattttgcaacttattactgtcaacagtttaatagttacccgtacacttttggccaggggaccaagctggagatcaaaggtggcggtggctcgggcggtggtgggtcgggtggcggcggatctgaggtgcagctggtgcagtctggagcagaggtgaaaaagcccggggagtctctgaagatctcctgtaagggttctggatacagctttagcagcagctggatcggctgggtgcgccagatgcccgggaaaggcctggagtggatggggatcatctatcctgatgactctgataccagatacagtccgtccttccaaggccaggtcaccatctcagccgacaagtccatcaggaccgcctacctgcagtggagcagcctgaaggcctcggacaccgccatgtattactgtgcgagacatgttactatgatttggggagttattattgacttctggggccagggaaccctggtcaccgtctcctca

SEQ ID NO: 13 - DNA, 1467 bp, Artificial Sequence
  GPC3-59TM CAR
atggccttaccagtgaccgccttgctcctgccgctggccttgctgctccacgccgccaggccggacgtggtgatgacccagagccctttatctttacccgttacacccggtgagcccgctagcatctcttgtagaagcagccagtctttagtgcacagcaacggcaacacatatttacactggtatttacagaagcccggtcagagcccccagctgctgatctacaaggtgagcaatcgtttctccggcgtgcccgatagattcagcggcagcggctccggaaccgacttcactttaaagatcagcagagtggaggccgaggacgtgggtgtgtactactgctcccagaacacccacgtgccccctacattcggtcaaggtaccaaactggagatcaaaggtggcggtggctcgggcggtggtgggtcgggtggcggcggatctcaagttcagctggtgcagagcggcgccgaggtcaaaaaacccggagccagcgtgaaggtgtcttgtaaagccagcggctacacctttaccgactatgagatgcactgggtgagacaagctcccggtcaaggtctggaatggatgggcgctttagaccccaagactggtgacacagcctactcccagaagttcaagggtcgtgtgactttaacagccgacgagtccaccagcacagcctacatggaactgagctctttaaggagcgaggacaccgccgtgtattactgcactcgtttctacagctacacctactggggccaaggtactttagtgacagtgagcagcccacatttggagcaggagcctacaaatttgagagaatcatctctaagcaaaatgtcctcagatctgcaaatgagaaattcacaagcgcacaggaattttcttgaagatggagaaagtgatggctttttaagatgcctctctcttaactctgggtggattttaactacaactcttgtcctctcggtgatggtattgctttggatttgttgtgcaaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 14 - DNA, 2785 bp, Artificial Sequence
  GPC3-59TM CAR-IRES-GFP
atggccttaccagtgaccgccttgctcctgccgctggccttgctgctccacgccgccaggccggacgtggtgatgacccagagccctttatctttacccgttacacccggtgagcccgctagcatctcttgtagaagcagccagtctttagtgcacagcaacggcaacacatatttacactggtatttacagaagcccggtcagagcccccagctgctgatctacaaggtgagcaatcgtttctccggcgtgcccgatagattcagcggcagcggctccggaaccgacttcactttaaagatcagcagagtggaggccgaggacgtgggtgtgtactactgctcccagaacacccacgtgccccctacattcggtcaaggtaccaaactggagatcaaaggtggcggtggctcgggcggtggtgggtcgggtggcggcggatctcaagttcagctggtgcagagcggcgccgaggtcaaaaaacccggagccagcgtgaaggtgtcttgtaaagccagcggctacacctttaccgactatgagatgcactgggtgagacaagctcccggtcaaggtctggaatggatgggcgctttagaccccaagactggtgacacagcctactcccagaagttcaagggtcgtgtgactttaacagccgacgagtccaccagcacagcctacatggaactgagctctttaaggagcgaggacaccgccgtgtattactgcactcgtttctacagctacacctactggggccaaggtactttagtgacagtgagcagcccacatttggagcaggagcctacaaatttgagagaatcatctctaagcaaaatgtcctcagatctgcaaatgagaaattcacaagcgcacaggaattttcttgaagatggagaaagtgatggctttttaagatgcctctctcttaactctgggtggattttaactacaactcttgtcctctcggtgatggtattgctttggatttgttgtgcaaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaacccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataagcttgccacaacccacaatctagaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 15 - DNA, 720 bp, Artificial Sequence
  GFP targeting moiety
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 16 - DNA, 66 bp, Artificial Sequence
  nucleotide sequence of CD8 signal peptide
atgctgctgctcgtgacctctctgctgctgtgcgaactgccccaccccgcctttctgctgattccc

SEQ ID NO: 17 - DNA, 57 bp, Artificial Sequence
  2A sequence
gctactaacttcagcctgctgaagcaggctggagacgtggaggagaaccctggacct

SEQ ID NO: 18 - DNA, 574 bp, Artificial Sequence
  IRES sequence
cccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataa

SEQ ID NO: 19 - DNA, 1470 bp, Artificial Sequence
  H63 CAR
atgctgctgctcgtgacctctctgctgctgtgcgaactgccccaccccgcctttctgctgattccccaagtgcagctcgtccaaagcggcggaggagtggtgcagcccggaagaagcctcagactcagctgtgctgccagcggagtcagcctccccgactacggagtgagctgggtgagacaagcccccggcaagggactggaatgggtcgccgtgatttggggcagcgagaccacctactacaactccgctctgaagtctagattcacaatctccagagataattccaaaaataccctctatctgcaaatgaactctctgagggccgaggacaccgccgtgtactactgtgctaagcactactactatggcggcagcttcgccatggattactggggacaaggcacactggtgaccgtctcctccggaagcaccagcggaagcggaaagcccggcagcggcgagggctccacaaaaggagacatccagatgacacaatccccttcctctctgagcgcttccgtcggcgacagagtcaccatcacatgcagagccagccaagacatcagcaagtatctgaactggtaccagcagaagcccggcaaggccatcaaactgctgatctaccacacatctagactgcacagcggagtgccctccagattctccggcagcggcagcggcaccgactttacactgaccattagctctctgcagcccgaggactttgctacctactactgtcagcaaggcgccacactgccctatacctttggccccggcacaaaggtggacatcaagaccacaacacccgcccctagacctcccacccccgcccctaccatcgctagccagcctctgtctctgagacccgaggcttgcagacccgctgctggaggagctgtgcatacaagaggactggatttcgcttgcgacatctacatctgggcccctctggctggcacatgtggagtgctgctgctgtctctggtgatcaccctctactgcaagagaggaagaaaaaaactgctctatatttttaaacagccttttatgaggcccgtccagaccacacaagaggaagacggatgtagctgcagattccccgaggaggaagagggaggatgcgagctgagagtcaagttctctagatccgctgatgcccccgcctacaaacaaggccagaatcagctctacaacgagctcaacctcggcagaagagaggagtacgacgtgctggacaagagaagaggcagagatcccgagatgggcggaaagcctagaaggaagaaccctcaagagggactgtacaacgagctgcagaaggacaaaatggctgaggcctacagcgaaatcggcatgaaaggcgagaggaggaggggcaaaggacacgatggactgtaccaaggcctctccaccgccacaaaggacacctacgacgccctccatatgcaagctctgccccctagg

SEQ ID NO: 20 - DNA, 2511 bp, Artificial Sequence
  H63 CAR-2A-TMEM59
atgctgctgctcgtgacctctctgctgctgtgcgaactgccccaccccgcctttctgctgattccccaagtgcagctcgtccaaagcggcggaggagtggtgcagcccggaagaagcctcagactcagctgtgctgccagcggagtcagcctccccgactacggagtgagctgggtgagacaagcccccggcaagggactggaatgggtcgccgtgatttggggcagcgagaccacctactacaactccgctctgaagtctagattcacaatctccagagataattccaaaaataccctctatctgcaaatgaactctctgagggccgaggacaccgccgtgtactactgtgctaagcactactactatggcggcagcttcgccatggattactggggacaaggcacactggtgaccgtctcctccggaagcaccagcggaagcggaaagcccggcagcggcgagggctccacaaaaggagacatccagatgacacaatccccttcctctctgagcgcttccgtcggcgacagagtcaccatcacatgcagagccagccaagacatcagcaagtatctgaactggtaccagcagaagcccggcaaggccatcaaactgctgatctaccacacatctagactgcacagcggagtgccctccagattctccggcagcggcagcggcaccgactttacactgaccattagctctctgcagcccgaggactttgctacctactactgtcagcaaggcgccacactgccctatacctttggccccggcacaaaggtggacatcaagaccacaacacccgcccctagacctcccacccccgcccctaccatcgctagccagcctctgtctctgagacccgaggcttgcagacccgctgctggaggagctgtgcatacaagaggactggatttcgcttgcgacatctacatctgggcccctctggctggcacatgtggagtgctgctgctgtctctggtgatcaccctctactgcaagagaggaagaaaaaaactgctctatatttttaaacagccttttatgaggcccgtccagaccacacaagaggaagacggatgtagctgcagattccccgaggaggaagagggaggatgcgagctgagagtcaagttctctagatccgctgatgcccccgcctacaaacaaggccagaatcagctctacaacgagctcaacctcggcagaagagaggagtacgacgtgctggacaagagaagaggcagagatcccgagatgggcggaaagcctagaaggaagaaccctcaagagggactgtacaacgagctgcagaaggacaaaatggctgaggcctacagcgaaatcggcatgaaaggcgagaggaggaggggcaaaggacacgatggactgtaccaaggcctctccaccgccacaaaggacacctacgacgccctccatatgcaagctctgccccctaggggaagcggagctactaacttcagcctgctgaagcaggctggagacgtggaggagaaccctggacctatggcggcgccgaaggggagcctctgggtgaggacccaactggggctcccgccgctgctgctgctgaccatggccttggccggaggttcggggaccgcttcggctgaagcatttgactcggtcttgggtgatacggcgtcttgccaccgggcctgtcagttgacctaccccttgcacacctaccctaaggaagaggagttgtacgcatgtcagagaggttgcaggctgttttcaatttgtcagtttgtggatgatggaattgacttaaatcgaactaaattggaatgtgaatctgcatgtacagaagcatattcccaatctgatgagcaatatgcttgccatcttggttgccagaatcagctgccattcgctgaactgagacaagaacaacttatgtccctgatgccaaaaatgcacctactctttcctctaactctggtgaggtcattctggagtgacatgatggactccgcacagagcttcataacctcttcatggactttttatcttcaagccgatgacggaaaaatagttatattccagtctaagccagaaatccagtacgcaccacatttggagcaggagcctacaaatttgagagaatcatctctaagcaaaatgtcctcagatctgcaaatgagaaattcacaagcgcacaggaattttcttgaagatggagaaagtgatggctttttaagatgcctctctcttaactctgggtggattttaactacaactcttgtcctctcggtgatggtattgctttggatttgttgtgcaactgttgctacagctgtggagcagtatgttccctctgagaagctgagtatctatggtgacttggagtttatgaatgaacaaaagctaaacagatatccagcttcttctcttgtggttgttagatctaaaactgaagatcatgaagaagcagggcctctacctacaaaagtgaatcttgctcattctgaaatttaa

SEQ ID NO: 21 - DNA, 1518 bp, Artificial Sequence
  21D4 CAR
atggccttaccagtgaccgccttgctcctgccgctggccttgctgctccacgccgccaggccggccatccagttgacccagtctccatcctccctgtctgcatctgtaggagacagagtcaccatcacttgccgggcaagtcagggcattagcagtgctttagcctggtatcagcagaaaccagggaaagctcctaagctcctgatctatgatgcctccagtttggaaagtggggtcccatcaaggttcagcggcagtggatctgggacagatttcactctcaccatcagcagcctgcagcctgaagattttgcaacttattactgtcaacagtttaatagttacccgtacacttttggccaggggaccaagctggagatcaaaggtggcggtggctcgggcggtggtgggtcgggtggcggcggatctgaggtgcagctggtgcagtctggagcagaggtgaaaaagcccggggagtctctgaagatctcctgtaagggttctggatacagctttagcagcagctggatcggctgggtgcgccagatgcccgggaaaggcctggagtggatggggatcatctatcctgatgactctgataccagatacagtccgtccttccaaggccaggtcaccatctcagccgacaagtccatcaggaccgcctacctgcagtggagcagcctgaaggcctcggacaccgccatgtattactgtgcgagacatgttactatgatttggggagttattattgacttctggggccagggaaccctggtcaccgtctcctcattcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaccacaggaaccgtttctctgttgttaaacggggcagaaagaagctcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 22 - DNA, 2682 bp, Artificial Sequence
  21D4 CAR-2A-TMEM59
atggccttaccagtgaccgccttgctcctgccgctggccttgctgctccacgccgccaggccggccatccagttgacccagtctccatcctccctgtctgcatctgtaggagacagagtcaccatcacttgccgggcaagtcagggcattagcagtgctttagcctggtatcagcagaaaccagggaaagctcctaagctcctgatctatgatgcctccagtttggaaagtggggtcccatcaaggttcagcggcagtggatctgggacagatttcactctcaccatcagcagcctgcagcctgaagattttgcaacttattactgtcaacagtttaatagttacccgtacacttttggccaggggaccaagctggagatcaaaggtggcggtggctcgggcggtggtgggtcgggtggcggcggatctgaggtgcagctggtgcagtctggagcagaggtgaaaaagcccggggagtctctgaagatctcctgtaagggttctggatacagctttagcagcagctggatcggctgggtgcgccagatgcccgggaaaggcctggagtggatggggatcatctatcctgatgactctgataccagatacagtccgtccttccaaggccaggtcaccatctcagccgacaagtccatcaggaccgcctacctgcagtggagcagcctgaaggcctcggacaccgccatgtattactgtgcgagacatgttactatgatttggggagttattattgacttctggggccagggaaccctggtcaccgtctcctcattcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaccacaggaacaggagtaagaggagcaggctcctgcacagtgactacatgaacatgactccccgccgccccgggcccacccgcaagcattaccagccctatgccccaccacgcgacttcgcagcctatcgctcccgtttctctgttgttaaacggggcagaaagaagctcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggaagcggagctactaacttcagcctgctgaagcaggctggagacgtggaggagaaccctggacctatggcggcgccgaaggggagcctctgggtgaggacccaactggggctcccgccgctgctgctgctgaccatggccttggccggaggttcggggaccgcttcggctgaagcatttgactcggtcttgggtgatacggcgtcttgccaccgggcctgtcagttgacctaccccttgcacacctaccctaaggaagaggagttgtacgcatgtcagagaggttgcaggctgttttcaatttgtcagtttgtggatgatggaattgacttaaatcgaactaaattggaatgtgaatctgcatgtacagaagcatattcccaatctgatgagcaatatgcttgccatcttggttgccagaatcagctgccattcgctgaactgagacaagaacaacttatgtccctgatgccaaaaatgcacctactctttcctctaactctggtgaggtcattctggagtgacatgatggactccgcacagagcttcataacctcttcatggactttttatcttcaagccgatgacggaaaaatagttatattccagtctaagccagaaatccagtacgcaccacatttggagcaggagcctacaaatttgagagaatcatctctaagcaaaatgtcctcagatctgcaaatgagaaattcacaagcgcacaggaattttcttgaagatggagaaagtgatggctttttaagatgcctctctcttaactctgggtggattttaactacaactcttgtcctctcggtgatggtattgctttggatttgttgtgcaactgttgctacagctgtggagcagtatgttccctctgagaagctgagtatctatggtgacttggagtttatgaatgaacaaaagctaaacagatatccagcttcttctcttgtggttgttagatctaaaactgaagatcatgaagaagcagggcctctacctacaaaagtgaatcttgctcattctgaaatttaa

SEQ ID NO: 23 - DNA, 2304 bp, Artificial Sequence
  21D4 CAR-2A-GFP
atggccttaccagtgaccgccttgctcctgccgctggccttgctgctccacgccgccaggccggccatccagttgacccagtctccatcctccctgtctgcatctgtaggagacagagtcaccatcacttgccgggcaagtcagggcattagcagtgctttagcctggtatcagcagaaaccagggaaagctcctaagctcctgatctatgatgcctccagtttggaaagtggggtcccatcaaggttcagcggcagtggatctgggacagatttcactctcaccatcagcagcctgcagcctgaagattttgcaacttattactgtcaacagtttaatagttacccgtacacttttggccaggggaccaagctggagatcaaaggtggcggtggctcgggcggtggtgggtcgggtggcggcggatctgaggtgcagctggtgcagtctggagcagaggtgaaaaagcccggggagtctctgaagatctcctgtaagggttctggatacagctttagcagcagctggatcggctgggtgcgccagatgcccgggaaaggcctggagtggatggggatcatctatcctgatgactctgataccagatacagtccgtccttccaaggccaggtcaccatctcagccgacaagtccatcaggaccgcctacctgcagtggagcagcctgaaggcctcggacaccgccatgtattactgtgcgagacatgttactatgatttggggagttattattgacttctggggccagggaaccctggtcaccgtctcctcattcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaccacaggaaccgtttctctgttgttaaacggggcagaaagaagctcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggaagcggagctactaacttcagcctgctgaagcaggctggagacgtggaggagaaccctggacctatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 24 - DNA, 2970 bp, Artificial Sequence
  21D4 CAR-IRES-GFP
atggccttaccagtgaccgccttgctcctgccgctggccttgctgctccacgccgccaggccggccatccagttgacccagtctccatcctccctgtctgcatctgtaggagacagagtcaccatcacttgccgggcaagtcagggcattagcagtgctttagcctggtatcagcagaaaccagggaaagctcctaagctcctgatctatgatgcctccagtttggaaagtggggtcccatcaaggttcagcggcagtggatctgggacagatttcactctcaccatcagcagcctgcagcctgaagattttgcaacttattactgtcaacagtttaatagttacccgtacacttttggccaggggaccaagctggagatcaaaggtggcggtggctcgggcggtggtgggtcgggtggcggcggatctgaggtgcagctggtgcagtctggagcagaggtgaaaaagcccggggagtctctgaagatctcctgtaagggttctggatacagctttagcagcagctggatcggctgggtgcgccagatgcccgggaaaggcctggagtggatggggatcatctatcctgatgactctgataccagatacagtccgtccttccaaggccaggtcaccatctcagccgacaagtccatcaggaccgcctacctgcagtggagcagcctgaaggcctcggacaccgccatgtattactgtgcgagacatgttactatgatttggggagttattattgacttctggggccagggaaccctggtcaccgtctcctcattcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaccacaggaacaggagtaagaggagcaggctcctgcacagtgactacatgaacatgactccccgccgccccgggcccacccgcaagcattaccagccctatgccccaccacgcgacttcgcagcctatcgctcccgtttctctgttgttaaacggggcagaaagaagctcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaaggatcccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataagcttgccacaacccacaatctagaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 25 - DNA, 1518 bp, Artificial Sequence
  GPC3 CAR
atggccttaccagtgaccgccttgctcctgccgctggccttgctgctccacgccgccaggccggacgtggtgatgacccagagccctttatctttacccgttacacccggtgagcccgctagcatctcttgtagaagcagccagtctttagtgcacagcaacggcaacacatatttacactggtatttacagaagcccggtcagagcccccagctgctgatctacaaggtgagcaatcgtttctccggcgtgcccgatagattcagcggcagcggctccggaaccgacttcactttaaagatcagcagagtggaggccgaggacgtgggtgtgtactactgctcccagaacacccacgtgccccctacattcggtcaaggtaccaaactggagatcaaaggtggcggtggctcgggcggtggtgggtcgggtggcggcggatctcaagttcagctggtgcagagcggcgccgaggtcaaaaaacccggagccagcgtgaaggtgtcttgtaaagccagcggctacacctttaccgactatgagatgcactgggtgagacaagctcccggtcaaggtctggaatggatgggcgctttagaccccaagactggtgacacagcctactcccagaagttcaagggtcgtgtgactttaacagccgacgagtccaccagcacagcctacatggaactgagctctttaaggagcgaggacaccgccgtgtattactgcactcgtttctacagctacacctactggggccaaggtactttagtgacagtgagcagcttcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaccacaggaaccgtttctctgttgttaaacggggcagaaagaagctcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaaaagaagaggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 26 - DNA, 3099 bp, Artificial Sequence
  GPC3 CAR-IRES-TMEM59
atggccttaccagtgaccgccttgctcctgccgctggccttgctgctccacgccgccaggccggacgtggtgatgacccagagccctttatctttacccgttacacccggtgagcccgctagcatctcttgtagaagcagccagtctttagtgcacagcaacggcaacacatatttacactggtatttacagaagcccggtcagagcccccagctgctgatctacaaggtgagcaatcgtttctccggcgtgcccgatagattcagcggcagcggctccggaaccgacttcactttaaagatcagcagagtggaggccgaggacgtgggtgtgtactactgctcccagaacacccacgtgccccctacattcggtcaaggtaccaaactggagatcaaaggtggcggtggctcgggcggtggtgggtcgggtggcggcggatctcaagttcagctggtgcagagcggcgccgaggtcaaaaaacccggagccagcgtgaaggtgtcttgtaaagccagcggctacacctttaccgactatgagatgcactgggtgagacaagctcccggtcaaggtctggaatggatgggcgctttagaccccaagactggtgacacagcctactcccagaagttcaagggtcgtgtgactttaacagccgacgagtccaccagcacagcctacatggaactgagctctttaaggagcgaggacaccgccgtgtattactgcactcgtttctacagctacacctactggggccaaggtactttagtgacagtgagcagcttcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaccacaggaaccgtttctctgttgttaaacggggcagaaagaagctcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaaaagaagaggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaaggatcccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataagcttgccacaacccacaatctagaatggcggcgccgaaggggagcctctgggtgaggacccaactggggctcccgccgctgctgctgctgaccatggccttggccggaggttcggggaccgcttcggctgaagcatttgactcggtcttgggtgatacggcgtcttgccaccgggcctgtcagttgacctaccccttgcacacctaccctaaggaagaggagttgtacgcatgtcagagaggttgcaggctgttttcaatttgtcagtttgtggatgatggaattgacttaaatcgaactaaattggaatgtgaatctgcatgtacagaagcatattcccaatctgatgagcaatatgcttgccatcttggttgccagaatcagctgccattcgctgaactgagacaagaacaacttatgtccctgatgccaaaaatgcacctactctttcctctaactctggtgaggtcattctggagtgacatgatggactccgcacagagcttcataacctcttcatggactttttatcttcaagccgatgacggaaaaatagttatattccagtctaagccagaaatccagtacgcaccacatttggagcaggagcctacaaatttgagagaatcatctctaagcaaaatgtcctcagatctgcaaatgagaaattcacaagcgcacaggaattttcttgaagatggagaaagtgatggctttttaagatgcctctctcttaactctgggtggattttaactacaactcttgtcctctcggtgatggtattgctttggatttgttgtgcaactgttgctacagctgtggagcagtatgttccctctgagaagctgagtatctatggtgacttggagtttatgaatgaacaaaagctaaacagatatccagcttcttctcttgtggttgttagatctaaaactgaagatcatgaagaagcagggcctctacctacaaaagtgaatcttgctcattctgaaatttaa

SEQ ID NO: 27 - DNA, 2844 bp, Artificial Sequence
  gpc3 car-ires-gfp
atggccttaccagtgaccgccttgctcctgccgctggccttgctgctccacgccgccaggccggacgtggtgatgacccagagccctttatctttacccgttacacccggtgagcccgctagcatctcttgtagaagcagccagtctttagtgcacagcaacggcaacacatatttacactggtatttacagaagcccggtcagagcccccagctgctgatctacaaggtgagcaatcgtttctccggcgtgcccgatagattcagcggcagcggctccggaaccgacttcactttaaagatcagcagagtggaggccgaggacgtgggtgtgtactactgctcccagaacacccacgtgccccctacattcggtcaaggtaccaaactggagatcaaaggtggcggtggctcgggcggtggtgggtcgggtggcggcggatctcaagttcagctggtgcagagcggcgccgaggtcaaaaaacccggagccagcgtgaaggtgtcttgtaaagccagcggctacacctttaccgactatgagatgcactgggtgagacaagctcccggtcaaggtctggaatggatgggcgctttagaccccaagactggtgacacagcctactcccagaagttcaagggtcgtgtgactttaacagccgacgagtccaccagcacagcctacatggaactgagctctttaaggagcgaggacaccgccgtgtattactgcactcgtttctacagctacacctactggggccaaggtactttagtgacagtgagcagcttcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaccacaggaaccgtttctctgttgttaaacggggcagaaagaagctcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaaaagaagaggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaaggatcccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataagcttgccacaacccacaatctagaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 28 - DNA, 2202 bp, Artificial Sequence
  CD19CD22 CAR
atgctgctgctcgtgacaagcctgctgctgtgcgagctgccccaccctgcctttctgctgatccccgacatccagatgacccagaccaccagcagcctgagcgccagcctgggcgatagagtgaccatcagctgcagagccagccaggacatcagcaagtacctgaactggtatcagcagaaacccgacggcaccgtgaagctgctgatctaccacaccagcagactgcacagcggcgtgcccagcagattttctggcagcggctccggcaccgactacagcctgaccatctccaacctggaacaggaagatatcgctacctacttctgtcagcaaggcaacaccctgccctacaccttcggcggaggcaccaagctggaaatcacaggcggcggaggatcccaggtgcagctgcagcagtctggacccggcctcgtgaagcctagccagaccctgtctctgacctgcgccatcagcggcgatagcgtgtccagcaatagcgccgcctggaactggatccggcagagcccttctagaggcctggaatggctgggccggacctactaccggtccaagtggtacaacgactacgccgtgtccgtgaagtcccggatcaccatcaaccccgacaccagcaagaaccagttctccctgcagctgaacagcgtgacccccgaggataccgccgtgtactactgcgccagagaagtgaccggcgacctggaagatgccttcgacatctggggccagggcacaatggtcaccgtgtctagcggcagcacaagcggctctggcaagcctggatctggcgagggctctaccaagggcgatattcagatgacacagagcccctccagcctgtccgcctctgtgggagacagagtgacaatcacctgtcgggcctcccagaccatctggtcctatctgaattggtatcagcagcggcctggcaaggcccccaacctgctgatctatgccgccagctctctgcagtccggcgtgccatctagattcagcggcagaggcagcggcaccgatttcaccctgacaattagcagtctgcaggccgaggacttcgccacctactattgccagcagagctacagcatcccccagaccttcggccagggaacaaaactggaaatcaaagggggaggcggcagcgaagtgaaactgcaggaatctggccctggcctggtggccccaagccagtctctgagcgtgacctgtaccgtgtctggcgtgtccctgcccgattacggcgtgtcctggatcagacagccccccagaaagggactggaatggctgggagtgatctggggcagcgagacaacctactacaacagcgccctgaagtccaggctgaccatcatcaaggacaactccaagagccaggtgttcctgaagatgaattccctgcagaccgacgacaccgccatctattactgtgccaagcactactactacggcggcagctacgccatggactactggggacagggaacctccgtgaccgtgtcctcttccggaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 29 - DNA, 3783 bp, Artificial Sequence
  CD19CD22 CAR-IRES-TMEM59
atgctgctgctcgtgacaagcctgctgctgtgcgagctgccccaccctgcctttctgctgatccccgacatccagatgacccagaccaccagcagcctgagcgccagcctgggcgatagagtgaccatcagctgcagagccagccaggacatcagcaagtacctgaactggtatcagcagaaacccgacggcaccgtgaagctgctgatctaccacaccagcagactgcacagcggcgtgcccagcagattttctggcagcggctccggcaccgactacagcctgaccatctccaacctggaacaggaagatatcgctacctacttctgtcagcaaggcaacaccctgccctacaccttcggcggaggcaccaagctggaaatcacaggcggcggaggatcccaggtgcagctgcagcagtctggacccggcctcgtgaagcctagccagaccctgtctctgacctgcgccatcagcggcgatagcgtgtccagcaatagcgccgcctggaactggatccggcagagcccttctagaggcctggaatggctgggccggacctactaccggtccaagtggtacaacgactacgccgtgtccgtgaagtcccggatcaccatcaaccccgacaccagcaagaaccagttctccctgcagctgaacagcgtgacccccgaggataccgccgtgtactactgcgccagagaagtgaccggcgacctggaagatgccttcgacatctggggccagggcacaatggtcaccgtgtctagcggcagcacaagcggctctggcaagcctggatctggcgagggctctaccaagggcgatattcagatgacacagagcccctccagcctgtccgcctctgtgggagacagagtgacaatcacctgtcgggcctcccagaccatctggtcctatctgaattggtatcagcagcggcctggcaaggcccccaacctgctgatctatgccgccagctctctgcagtccggcgtgccatctagattcagcggcagaggcagcggcaccgatttcaccctgacaattagcagtctgcaggccgaggacttcgccacctactattgccagcagagctacagcatcccccagaccttcggccagggaacaaaactggaaatcaaagggggaggcggcagcgaagtgaaactgcaggaatctggccctggcctggtggccccaagccagtctctgagcgtgacctgtaccgtgtctggcgtgtccctgcccgattacggcgtgtcctggatcagacagccccccagaaagggactggaatggctgggagtgatctggggcagcgagacaacctactacaacagcgccctgaagtccaggctgaccatcatcaaggacaactccaagagccaggtgttcctgaagatgaattccctgcagaccgacgacaccgccatctattactgtgccaagcactactactacggcggcagctacgccatggactactggggacagggaacctccgtgaccgtgtcctcttccggaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaaggatcccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataagcttgccacaacccacaatctagaatggcggcgccgaaggggagcctctgggtgaggacccaactggggctcccgccgctgctgctgctgaccatggccttggccggaggttcggggaccgcttcggctgaagcatttgactcggtcttgggtgatacggcgtcttgccaccgggcctgtcagttgacctaccccttgcacacctaccctaaggaagaggagttgtacgcatgtcagagaggttgcaggctgttttcaatttgtcagtttgtggatgatggaattgacttaaatcgaactaaattggaatgtgaatctgcatgtacagaagcatattcccaatctgatgagcaatatgcttgccatcttggttgccagaatcagctgccattcgctgaactgagacaagaacaacttatgtccctgatgccaaaaatgcacctactctttcctctaactctggtgaggtcattctggagtgacatgatggactccgcacagagcttcataacctcttcatggactttttatcttcaagccgatgacggaaaaatagttatattccagtctaagccagaaatccagtacgcaccacatttggagcaggagcctacaaatttgagagaatcatctctaagcaaaatgtcctcagatctgcaaatgagaaattcacaagcgcacaggaattttcttgaagatggagaaagtgatggctttttaagatgcctctctcttaactctgggtggattttaactacaactcttgtcctctcggtgatggtattgctttggatttgttgtgcaactgttgctacagctgtggagcagtatgttccctctgagaagctgagtatctatggtgacttggagtttatgaatgaacaaaagctaaacagatatccagcttcttctcttgtggttgttagatctaaaactgaagatcatgaagaagcagggcctctacctacaaaagtgaatcttgctcattctgaaatttaa

SEQ ID NO: 30 - DNA, 3528 bp, Artificial Sequence
  CD19CD22 CAR-IRES-GFP
atgctgctgctcgtgacaagcctgctgctgtgcgagctgccccaccctgcctttctgctgatccccgacatccagatgacccagaccaccagcagcctgagcgccagcctgggcgatagagtgaccatcagctgcagagccagccaggacatcagcaagtacctgaactggtatcagcagaaacccgacggcaccgtgaagctgctgatctaccacaccagcagactgcacagcggcgtgcccagcagattttctggcagcggctccggcaccgactacagcctgaccatctccaacctggaacaggaagatatcgctacctacttctgtcagcaaggcaacaccctgccctacaccttcggcggaggcaccaagctggaaatcacaggcggcggaggatcccaggtgcagctgcagcagtctggacccggcctcgtgaagcctagccagaccctgtctctgacctgcgccatcagcggcgatagcgtgtccagcaatagcgccgcctggaactggatccggcagagcccttctagaggcctggaatggctgggccggacctactaccggtccaagtggtacaacgactacgccgtgtccgtgaagtcccggatcaccatcaaccccgacaccagcaagaaccagttctccctgcagctgaacagcgtgacccccgaggataccgccgtgtactactgcgccagagaagtgaccggcgacctggaagatgccttcgacatctggggccagggcacaatggtcaccgtgtctagcggcagcacaagcggctctggcaagcctggatctggcgagggctctaccaagggcgatattcagatgacacagagcccctccagcctgtccgcctctgtgggagacagagtgacaatcacctgtcgggcctcccagaccatctggtcctatctgaattggtatcagcagcggcctggcaaggcccccaacctgctgatctatgccgccagctctctgcagtccggcgtgccatctagattcagcggcagaggcagcggcaccgatttcaccctgacaattagcagtctgcaggccgaggacttcgccacctactattgccagcagagctacagcatcccccagaccttcggccagggaacaaaactggaaatcaaagggggaggcggcagcgaagtgaaactgcaggaatctggccctggcctggtggccccaagccagtctctgagcgtgacctgtaccgtgtctggcgtgtccctgcccgattacggcgtgtcctggatcagacagccccccagaaagggactggaatggctgggagtgatctggggcagcgagacaacctactacaacagcgccctgaagtccaggctgaccatcatcaaggacaactccaagagccaggtgttcctgaagatgaattccctgcagaccgacgacaccgccatctattactgtgccaagcactactactacggcggcagctacgccatggactactggggacagggaacctccgtgaccgtgtcctcttccggaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaaggatcccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataagcttgccacaacccacaatctagaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 31 - DNA, 2202 bp, Artificial Sequence
  CD19CD22-59TM CAR
atgctgctgctggtgacatctctgctcctctgcgaactccctcatcccgcctttctgctgatccccgacatccagatgacacagaccacatcctctctgtccgcctctctgggagatagggtgacaatcagctgtagagcttcccaagacatctccaagtatctgaactggtaccagcagaaacccgatggaaccgtcaaactgctgatttaccatacctctagactgcactccggagtgccttccagatttagcggcagcggcagcggcacagactacagcctcaccatctccaatctggagcaagaggacattgctacctacttctgtcagcaaggcaacacactgccttacaccttcggcggcggcaccaaactggagatcaccggcggcggaggaagccaagtgcagctgcagcagagcggccccggcctcgtgaaacccagccagaccctctctctgacatgcgctatttccggcgatagcgtgagctccaactccgccgcttggaactggattaggcagagcccctctagaggactggaatggctgggaaggacctactatagaagcaaatggtacaacgactacgccgtctccgtgaagtccagaatcaccatcaatcccgacacatccaagaatcagttctccctccagctgaacagcgtcacacccgaggacaccgccgtgtactactgcgctagagaggtgaccggcgatctggaggacgctttcgacatctggggccaaggaacaatggtcaccgtcagcagcggaagcacaagcggcagcggcaaacccggaagcggagagggcagcaccaagggagacattcaaatgacccagtcccccagctctctgagcgcttccgtgggagatagagtgaccattacatgcagagccagccagacaatctggtcctatctgaactggtatcagcagaggcccggaaaagcccccaatctgctgatctacgccgctagctctctgcaatccggagtgccctctagattttccggaaggggaagcggaaccgacttcaccctcaccatttcctctctgcaagccgaggatttcgctacatactactgccagcagtcctattccattccccagacctttggccaaggcaccaagctggaaattaaaggcggaggaggctccgaggtcaagctccaagaaagcggacccggactggtcgctcctagccagagcctcagcgtcacatgcaccgtgtccggagtgtccctccccgactacggagtgagctggattagacagccccctaggaaaggactggagtggctgggcgtgatctggggctccgagaccacctactacaatagcgctctgaagtctagactcacaatcattaaggacaacagcaaatcccaagtgttcctcaagatgaatagcctccagaccgacgatacagccatctattactgcgccaagcactactactacggcggcagctacgccatggactattggggacaaggcacatccgtcacagtcagcagcccacatttggagcaggagcctacaaatttgagagaatcatctctaagcaaaatgtcctcagatctgcaaatgagaaattcacaagcgcacaggaattttcttgaagatggagaaagtgatggctttttaagatgcctctctcttaactctgggtggattttaactacaactcttgtcctctcggtgatggtattgctttggatttgttgtgcaaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 32 - DNA, 3528 bp, Artificial Sequence
  CD19CD22-59TM CAR-IRES-GFP
atgctgctgctggtgacatctctgctcctctgcgaactccctcatcccgcctttctgctgatccccgacatccagatgacacagaccacatcctctctgtccgcctctctgggagatagggtgacaatcagctgtagagcttcccaagacatctccaagtatctgaactggtaccagcagaaacccgatggaaccgtcaaactgctgatttaccatacctctagactgcactccggagtgccttccagatttagcggcagcggcagcggcacagactacagcctcaccatctccaatctggagcaagaggacattgctacctacttctgtcagcaaggcaacacactgccttacaccttcggcggcggcaccaaactggagatcaccggcggcggaggaagccaagtgcagctgcagcagagcggccccggcctcgtgaaacccagccagaccctctctctgacatgcgctatttccggcgatagcgtgagctccaactccgccgcttggaactggattaggcagagcccctctagaggactggaatggctgggaaggacctactatagaagcaaatggtacaacgactacgccgtctccgtgaagtccagaatcaccatcaatcccgacacatccaagaatcagttctccctccagctgaacagcgtcacacccgaggacaccgccgtgtactactgcgctagagaggtgaccggcgatctggaggacgctttcgacatctggggccaaggaacaatggtcaccgtcagcagcggaagcacaagcggcagcggcaaacccggaagcggagagggcagcaccaagggagacattcaaatgacccagtcccccagctctctgagcgcttccgtgggagatagagtgaccattacatgcagagccagccagacaatctggtcctatctgaactggtatcagcagaggcccggaaaagcccccaatctgctgatctacgccgctagctctctgcaatccggagtgccctctagattttccggaaggggaagcggaaccgacttcaccctcaccatttcctctctgcaagccgaggatttcgctacatactactgccagcagtcctattccattccccagacctttggccaaggcaccaagctggaaattaaaggcggaggaggctccgaggtcaagctccaagaaagcggacccggactggtcgctcctagccagagcctcagcgtcacatgcaccgtgtccggagtgtccctccccgactacggagtgagctggattagacagccccctaggaaaggactggagtggctgggcgtgatctggggctccgagaccacctactacaatagcgctctgaagtctagactcacaatcattaaggacaacagcaaatcccaagtgttcctcaagatgaatagcctccagaccgacgatacagccatctattactgcgccaagcactactactacggcggcagctacgccatggactattggggacaaggcacatccgtcacagtcagcagcccacatttggagcaggagcctacaaatttgagagaatcatctctaagcaaaatgtcctcagatctgcaaatgagaaattcacaagcgcacaggaattttcttgaagatggagaaagtgatggctttttaagatgcctctctcttaactctgggtggattttaactacaactcttgtcctctcggtgatggtattgctttggatttgttgtgcaaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaaggatcccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataagcttgccacaacccacaatctagaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 33 - DNA, 1470 bp, Artificial Sequence
  21D4-59TM CAR
atggccttaccagtgaccgccttgctcctgccgctggccttgctgctccacgccgccaggccggccatccagttgacccagtctccatcctccctgtctgcatctgtaggagacagagtcaccatcacttgccgggcaagtcagggcattagcagtgctttagcctggtatcagcagaaaccagggaaagctcctaagctcctgatctatgatgcctccagtttggaaagtggggtcccatcaaggttcagcggcagtggatctgggacagatttcactctcaccatcagcagcctgcagcctgaagattttgcaacttattactgtcaacagtttaatagttacccgtacacttttggccaggggaccaagctggagatcaaaggtggcggtggctcgggcggtggtgggtcgggtggcggcggatctgaggtgcagctggtgcagtctggagcagaggtgaaaaagcccggggagtctctgaagatctcctgtaagggttctggatacagctttagcagcagctggatcggctgggtgcgccagatgcccgggaaaggcctggagtggatggggatcatctatcctgatgactctgataccagatacagtccgtccttccaaggccaggtcaccatctcagccgacaagtccatcaggaccgcctacctgcagtggagcagcctgaaggcctcggacaccgccatgtattactgtgcgagacatgttactatgatttggggagttattattgacttctggggccagggaaccctggtcaccgtctcctcaccacatttggagcaggagcctacaaatttgagagaatcatctctaagcaaaatgtcctcagatctgcaaatgagaaattcacaagcgcacaggaattttcttgaagatggagaaagtgatggctttttaagatgcctctctcttaactctgggtggattttaactacaactcttgtcctctcggtgatggtattgctttggatttgttgtgcaaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 34 - DNA, 252 bp, Artificial Sequence
  nucleotide sequence of CD8 transmembrane region
ttcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaccacaggaacagg

SEQ ID NO: 35 - DNA, 747 bp, Artificial Sequence
  CD22 targeting moiety
caagtgcagctgcagcagtccggacccggcctcgtcaagccctcccagaccctctctctgacatgtgccatcagcggcgatagcgtcagctccaacagcgctgcttggaactggattaggcagagccctagcagaggactggagtggctgggaaggacctactatagaagcaagtggtacaacgattacgccgtgagcgtcaagtctagaatcaccattaaccccgacacctccaagaaccagttctctctgcagctcaacagcgtgacccccgaggacaccgctgtctactattgtgccagagaggtgaccggagatctggaggatgccttcgacatctggggccaaggcaccatggtcaccgtgagcagcggctccacatccggctccggaaagcccggcagcggcgaaggctccacaaagggcgacatccagatgacccaaagcccttcctctctgtccgctagcgtcggagatagagtcaccatcacatgcagagctagccagaccatttggagctacctcaactggtaccagcagaggcccggcaaggcccctaatctgctgatctacgctgcctcctctctgcagagcggagtgcctagcagattcagcggaaggggcagcggaaccgactttacactgaccattagcagcctccaagccgaagacttcgccacctactactgccaacagtcctactccattcctcagacatttggccaaggcaccaagctcgagatcaag

SEQ ID NO: 36 - DNA, 789 bp, Artificial Sequence
  GPC3 targeting moiety
atggccttaccagtgaccgccttgctcctgccgctggccttgctgctccacgccgccaggccggacgtggtgatgacccagagccctttatctttacccgttacacccggtgagcccgctagcatctcttgtagaagcagccagtctttagtgcacagcaacggcaacacatatttacactggtatttacagaagcccggtcagagcccccagctgctgatctacaaggtgagcaatcgtttctccggcgtgcccgatagattcagcggcagcggctccggaaccgacttcactttaaagatcagcagagtggaggccgaggacgtgggtgtgtactactgctcccagaacacccacgtgccccctacattcggtcaaggtaccaaactggagatcaaaggtggcggtggctcgggcggtggtgggtcgggtggcggcggatctcaagttcagctggtgcagagcggcgccgaggtcaaaaaacccggagccagcgtgaaggtgtcttgtaaagccagcggctacacctttaccgactatgagatgcactgggtgagacaagctcccggtcaaggtctggaatggatgggcgctttagaccccaagactggtgacacagcctactcccagaagttcaagggtcgtgtgactttaacagccgacgagtccaccagcacagcctacatggaactgagctctttaaggagcgaggacaccgccgtgtattactgcactcgtttctacagctacacctactggggccaaggtactttagtgacagtgagcagc

SEQ ID NO: 37 - protein, 84 aa, Artificial Sequence
  amino acid sequence of CD8 transmembrane region
FVPVFLPAKPTTTPAPRPPTPAPTIASQPLSLRPEACRPAAGGAVHTRGLDFACDIYIWAPLAGTCGVLLLSLVITLYCNHRNR

SEQ ID NO: 38 - protein, 45 aa, Artificial Sequence
  amino acid sequence of CD8 hinge region
TTTPAPRPPTPAPTIASQPLSLRPEACRPAAGGAVHTRGLDFACD

SEQ ID NO: 39 - protein, 112 aa, Artificial Sequence
  amino acid sequence of CD3 zeta signaling domain
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 40 - protein, 47 aa, Artificial Sequence
  amino acid sequence of human 4-1BB costimulatory domain
RFSVVKRGRKKLLYIFKQPFMRPVQTTQEEDGCSCRFPEEEEGGCEL

SEQ ID NO: 41 - protein, 245 aa, Artificial Sequence
  humanized CD19 monoclonal antibody single chain variable region
QVQLVQSGGGVVQPGRSLRLSCAASGVSLPDYGVSWVRQAPGKGLEWVAVIWGSETTYYNSALKSRFTISRDNSKNTLYLQMNSLRAEDTAVYYCAKHYYYGGSFAMDYWGQGTLVTVSSGSTSGSGKPGSGEGSTKGDIQMTQSPSSLSASVGDRVTITCRASQDISKYLNWYQQKPGKAIKLLIYHTSRLHSGVPSRFSGSGSGTDFTLTISSLQPEDFATYYCQQGATLPYTFGPGTKVDIK

SEQ ID NO: 42 - protein, 264 aa, Artificial Sequence
  full-humanized CD19 monoclonal single chain variable region 21D4
MALPVTALLLPLALLLHAARPAIQLTQSPSSLSASVGDRVTITCRASQGISSALAWYQQKPGKAPKLLIYDASSLESGVPSRFSGSGSGTDFTLTISSLQPEDFATYYCQQFNSYPYTFGQGTKLEIKGGGGSGGGGSGGGGSEVQLVQSGAEVKKPGESLKISCKGSGYSFSSSWIGWVRQMPGKGLEWMGIIYPDDSDTRYSPSFQGQVTISADKSIRTAYLQWSSLKASDTAMYYCARHVTMIWGVIIDFWGQGTLVTVSS

SEQ ID NO: 43 - protein, 488 aa, Artificial Sequence
  GPC3-59TM CAR
MALPVTALLLPLALLLHAARPDVVMTQSPLSLPVTPGEPASISCRSSQSLVHSNGNTYLHWYLQKPGQSPQLLIYKVSNRFSGVPDRFSGSGSGTDFTLKISRVEAEDVGVYYCSQNTHVPPTFGQGTKLEIKGGGGSGGGGSGGGGSQVQLVQSGAEVKKPGASVKVSCKASGYTFTDYEMHWVRQAPGQGLEWMGALDPKTGDTAYSQKFKGRVTLTADESTSTAYMELSSLRSEDTAVYYCTRFYSYTYWGQGTLVTVSSPHLEQEPTNLRESSLSKMSSDLQMRNSQAHRNFLEDGESDGFLRCLSLNSGWILTTTLVLSVMVLLWICCAKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 44 - protein, 489 aa, Artificial Sequence
  21D4-59TM CAR
MALPVTALLLPLALLLHAARPAIQLTQSPSSLSASVGDRVTITCRASQGISSALAWYQQKPGKAPKLLIYDASSLESGVPSRFSGSGSGTDFTLTISSLQPEDFATYYCQQFNSYPYTFGQGTKLEIKGGGGSGGGGSGGGGSEVQLVQSGAEVKKPGESLKISCKGSGYSFSSSWIGWVRQMPGKGLEWMGIIYPDDSDTRYSPSFQGQVTISADKSIRTAYLQWSSLKASDTAMYYCARHVTMIWGVIIDFWGQGTLVTVSSPHLEQEPTNLRESSLSKMSSDLQMRNSQAHRNFLEDGESDGFLRCLSLNSGWILTTTLVLSVMVLLWICCAKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 45 - protein, 239 aa, Artificial Sequence
  GFP targeting moiety
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 46 - protein, 22 aa, Artificial Sequence
  amino acid sequence of CD8 signal peptide
MLLLVTSLLLCELPHPAFLLIP

SEQ ID NO: 47 - protein, 19 aa, Artificial Sequence
  2A sequence
ATNFSLLKQAGDVEENPGP

SEQ ID NO: 48 - protein, 249 aa, Artificial Sequence
  CD22 targeting moiety
QVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGSTSGSGKPGSGEGSTKGDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIK

SEQ ID NO: 49 - protein, 490 aa, Artificial Sequence
  H63 CAR
MLLLVTSLLLCELPHPAFLLIPQVQLVQSGGGVVQPGRSLRLSCAASGVSLPDYGVSWVRQAPGKGLEWVAVIWGSETTYYNSALKSRFTISRDNSKNTLYLQMNSLRAEDTAVYYCAKHYYYGGSFAMDYWGQGTLVTVSSGSTSGSGKPGSGEGSTKGDIQMTQSPSSLSASVGDRVTITCRASQDISKYLNWYQQKPGKAIKLLIYHTSRLHSGVPSRFSGSGSGTDFTLTISSLQPEDFATYYCQQGATLPYTFGPGTKVDIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 50 - protein, 836 aa, Artificial Sequence
  H63 CAR-2A-TMEM59
MLLLVTSLLLCELPHPAFLLIPQVQLVQSGGGVVQPGRSLRLSCAASGVSLPDYGVSWVRQAPGKGLEWVAVIWGSETTYYNSALKSRFTISRDNSKNTLYLQMNSLRAEDTAVYYCAKHYYYGGSFAMDYWGQGTLVTVSSGSTSGSGKPGSGEGSTKGDIQMTQSPSSLSASVGDRVTITCRASQDISKYLNWYQQKPGKAIKLLIYHTSRLHSGVPSRFSGSGSGTDFTLTISSLQPEDFATYYCQQGATLPYTFGPGTKVDIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMAAPKGSLWVRTQLGLPPLLLLTMALAGGSGTASAEAFDSVLGDTASCHRACQLTYPLHTYPKEEELYACQRGCRLFSICQFVDDGIDLNRTKLECESACTEAYSQSDEQYACHLGCQNQLPFAELRQEQLMSLMPKMHLLFPLTLVRSFWSDMMDSAQSFITSSWTFYLQADDGKIVIFQSKPEIQYAPHLEQEPTNLRESSLSKMSSDLQMRNSQAHRNFLEDGESDGFLRCLSLNSGWILTTTLVLSVMVLLWICCATVATAVEQYVPSEKLSIYGDLEFMNEQKLNRYPASSLVVVRSKTEDHEEAGPLPTKVNLAHSEI

SEQ ID NO: 51 - protein, 506 aa, Artificial Sequence
  21D4 CAR
MALPVTALLLPLALLLHAARPAIQLTQSPSSLSASVGDRVTITCRASQGISSALAWYQQKPGKAPKLLIYDASSLESGVPSRFSGSGSGTDFTLTISSLQPEDFATYYCQQFNSYPYTFGQGTKLEIKGGGGSGGGGSGGGGSEVQLVQSGAEVKKPGESLKISCKGSGYSFSSSWIGWVRQMPGKGLEWMGIIYPDDSDTRYSPSFQGQVTISADKSIRTAYLQWSSLKASDTAMYYCARHVTMIWGVIIDFWGQGTLVTVSSFVPVFLPAKPTTTPAPRPPTPAPTIASQPLSLRPEACRPAAGGAVHTRGLDFACDIYIWAPLAGTCGVLLLSLVITLYCNHRNRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 52 - protein, 893 aa, Artificial Sequence
  21D4 CAR-2A-TMEM59
MALPVTALLLPLALLLHAARPAIQLTQSPSSLSASVGDRVTITCRASQGISSALAWYQQKPGKAPKLLIYDASSLESGVPSRFSGSGSGTDFTLTISSLQPEDFATYYCQQFNSYPYTFGQGTKLEIKGGGGSGGGGSGGGGSEVQLVQSGAEVKKPGESLKISCKGSGYSFSSSWIGWVRQMPGKGLEWMGIIYPDDSDTRYSPSFQGQVTISADKSIRTAYLQWSSLKASDTAMYYCARHVTMIWGVIIDFWGQGTLVTVSSFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMAAPKGSLWVRTQLGLPPLLLLTMALAGGSGTASAEAFDSVLGDTASCHRACQLTYPLHTYPKEEELYACQRGCRLFSICQFVDDGIDLNRTKLECESACTEAYSQSDEQYACHLGCQNQLPFAELRQEQLMSLMPKMHLLFPLTLVRSFWSDMMDSAQSFITSSWTFYLQADDGKIVIFQSKPEIQYAPHLEQEPTNLRESSLSKMSSDLQMRNSQAHRNFLEDGESDGFLRCLSLNSGWILTTTLVLSVMVLLWICCATVATAVEQYVPSEKLSIYGDLEFMNEQKLNRYPASSLVVVRSKTEDHEEAGPLPTKVNLAHSEI

SEQ ID NO: 53 - protein, 767 aa, Artificial Sequence
  21D4 CAR-2A-GFP
MALPVTALLLPLALLLHAARPAIQLTQSPSSLSASVGDRVTITCRASQGISSALAWYQQKPGKAPKLLIYDASSLESGVPSRFSGSGSGTDFTLTISSLQPEDFATYYCQQFNSYPYTFGQGTKLEIKGGGGSGGGGSGGGGSEVQLVQSGAEVKKPGESLKISCKGSGYSFSSSWIGWVRQMPGKGLEWMGIIYPDDSDTRYSPSFQGQVTISADKSIRTAYLQWSSLKASDTAMYYCARHVTMIWGVIIDFWGQGTLVTVSSFVPVFLPAKPTTTPAPRPPTPAPTIASQPLSLRPEACRPAAGGAVHTRGLDFACDIYIWAPLAGTCGVLLLSLVITLYCNHRNRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 54 - protein, 263 aa, Artificial Sequence
  GPC3 targeting moiety
MALPVTALLLPLALLLHAARPDVVMTQSPLSLPVTPGEPASISCRSSQSLVHSNGNTYLHWYLQKPGQSPQLLIYKVSNRFSGVPDRFSGSGSGTDFTLKISRVEAEDVGVYYCSQNTHVPPTFGQGTKLEIKGGGGSGGGGSGGGGSQVQLVQSGAEVKKPGASVKVSCKASGYTFTDYEMHWVRQAPGQGLEWMGALDPKTGDTAYSQKFKGRVTLTADESTSTAYMELSSLRSEDTAVYYCTRFYSYTYWGQGTLVTVSS

SEQ ID NO: 55 - protein, 505 aa, Artificial Sequence
  GPC3 CAR
MALPVTALLLPLALLLHAARPDVVMTQSPLSLPVTPGEPASISCRSSQSLVHSNGNTYLHWYLQKPGQSPQLLIYKVSNRFSGVPDRFSGSGSGTDFTLKISRVEAEDVGVYYCSQNTHVPPTFGQGTKLEIKGGGGSGGGGSGGGGSQVQLVQSGAEVKKPGASVKVSCKASGYTFTDYEMHWVRQAPGQGLEWMGALDPKTGDTAYSQKFKGRVTLTADESTSTAYMELSSLRSEDTAVYYCTRFYSYTYWGQGTLVTVSSFVPVFLPAKPTTTPAPRPPTPAPTIASQPLSLRPEACRPAAGGAVHTRGLDFACDIYIWAPLAGTCGVLLLSLVITLYCNHRNRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 56 - protein, 733 aa, Artificial Sequence
  CD19CD22 CAR
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGSTSGSGKPGSGEGSTKGDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIKGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSSGTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 57 - protein, 733 aa, Artificial Sequence
  CD19CD22-59TM CAR
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGSTSGSGKPGSGEGSTKGDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIKGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSPHLEQEPTNLRESSLSKMSSDLQMRNSQAHRNFLEDGESDGFLRCLSLNSGWILTTTLVLSVMVLLWICCAKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

(19) United States
(12) Patent Application Publication
(10) Pub. No.: US 2022/0380433 A1
(43) Pub. Date: Dec. 1, 2022

(54) TMEM59 PROTEIN DIMER OR CHIMERIC EXPRESSION RECEPTOR IMPROVING T CELL FUNCTION

(71) Applicant: SHANGHAI PHARMACEUTICALS HOLDING CO., LTD., Shanghai (CN)

(72) Inventors: Xuemei LIAO, Shanghai (CN); Jingwei HUANG, Shanghai (CN); Zhuo CHEN, Shanghai (CN); Yang LIU, Shanghai (CN); Xiaocui CHENG, Shanghai (CN); Manman XIE, Shanghai (CN); Rongfang SUN, Shanghai (CN); Yingying LI, Shanghai (CN); Guangxin XIA, Shanghai (CN); Ying KE, Shanghai (CN); Yanjun LIU, Shanghai (CN)

(73) Assignee: SHANGHAI PHARMACEUTICALS HOLDING CO., LTD., Shanghai (CN)

(21) Appl. No.: 17/776,537

(22) PCT Filed: Nov. 12, 2020
(86) PCT No.: PCT/CN2020/128440

§ 371 Date: May 12, 2022

(30) Foreign Application Priority Data
Nov. 13, 2019 (CN) 201911107366.8

(51) Int. Cl. C07K 14/705; C07K 14/725; A61P 35/00; A61K 35/17; C07K 16/28

(52) U.S. Cl. CPC C07K 14/705; C07K 14/70517; C07K 14/7051; C07K 14/70578; A61P 35/00; A61K 35/17; C07K 16/2803; C07K 2319/03; C07K 2319/02; C07K 2319/33; A61K 38/00

(57) ABSTRACT

The present invention relates to an immune cell, which contains a TMEM59 protein and/or a functional fragment thereof, a chimeric antigen receptor (CAR) and/or a coding element thereof, as well as a use of the immune cell in the preparation of a drug for treating tumors. Further provided are a method for promoting the proliferation of immune cells and a method for promoting the production of memory cells.

### FIELD OF THE INVENTION

The present application relates to the field of biomedicine, and specifically to a use of a chimeric antigen receptor T lymphocyte in the preparation of a composition for treating tumors and a method therefor.

### BACKGROUND OF THE INVENTION

Malignant tumor is a major disease that seriously threatens human life. Currently, there are no effective cures for most malignant tumors, which have disease recurrence and high mortality rates, thus seriously threatening the health of patients and significantly reducing the quality of life.

The chimeric antigen receptor T lymphocyte therapy is an adoptive immunotherapy emerged in recent years. A single-chain antibody variable region derived from a monoclonal antibody targeting a tumor specific antigen, together with a co-stimulatory molecule (e.g., 4-1BB, CD28 protein, etc.) and an intracellular segment CD3zeta of a T lymphocyte signaling region, forms a chimeric antigen receptor protein, which is expressed across the cell membrane on the surface of the T lymphocyte. When the single-chain antibody variable region recognizes the specific antigen on the surface of tumor cells, it activates the co-stimulatory molecule and CD3zeta, transmits the activation signals of the T lymphocyte, directly activates the first and second signals on which the activation of T lymphocyte depends, promotes T lymphocyte activation, t lymphocyte proliferation and enhances its immune killing function so as to achieve the immunomodulation and killing on the tumor cells, thereby realizing the purpose of removing the tumor.

Increasing the survival time of effector T lymphocytes in a patient is an important approach to optimize the therapeutic efficacy and prolong the effective time of the chimeric antigen receptor T therapy. It is demonstrated from clinical trials that the long-term disease remission obtained from treatment with the chimeric antigen receptor T lymphocyte therapy is closely related to the long-term survival and persistence of chimeric antigen receptor T lymphocytes in the patient. Although an effector T lymphocyte has a potent ability to lyse tumors, its survival time in the patient is relatively short. However, memory T lymphocytes, including effector memory T lymphocytes and central memory T lymphocytes, are able to expand for a long time in vivo and possess a potent proliferative ability, at the same time, they have the potential to differentiate into effector T lymphocytes, so that they can provide effector T lymphocytes continuously.

Therefore, chimeric antigen receptor T lymphocytes with extended survival time are urgently needed to maintain their efficacy.

### SUMMARY OF THE INVENTION

The present application provides an immune cell, the immune cell includes a TMEM59 protein and/or a functional fragment thereof and a chimeric antigen receptor (CAR) and/or a coding element thereof, as well as a use of the immune cell in the preparation of a drug for treating tumors. The present application further provides a method for promoting the proliferation of immune cells and a method for promoting the production of memory cells. The immune cell of the present application has one of the following beneficial effects: (1) capable of increasing the number of memory lymphocytes, for example, compared with CAR T cells not including the TMEM59 protein and/or the functional fragment thereof, by at least 50%, 60%, 70%, 80%, 90%, 100%, 150% or 200% above; (2) capable of enhancing the proliferative ability of lymphocytes, for example, compared with CAR T cells not including the TMEM59 protein and/or the functional fragment thereof, by at least 10%, 20%, 30%, 40% or 50% above; (3) enhancing the killing rate of lymphocytes against target cells, for example, compared with CAR T cells not including the TMEM59 protein and/or the functional fragment thereof, by at least 10%, 20%, 30% or higher; and, (4) inhibiting the growth of tumor cells, and/or improving the survival rate of subjects with tumors.

In one aspect, the present application provides an immune cell, which includes a TMEM59 protein and/or a functional fragment thereof located on the cell membrane, wherein the functional fragment includes at least a transmembrane region and a hinge region of the TMEM59 protein.

In some embodiments, the immune cell is modified to include the TMEM59 protein and/or the functional fragment thereof on the cell membrane.

In some embodiments, the TMEM59 protein and/or the functional fragment thereof is a human TMEM59 protein and/or a functional fragment thereof.

In some embodiments, the transmembrane region of the TMEM59 protein includes an amino acid sequence as set forth in SEQ ID NO. 1.

In some embodiments, the hinge region of the TMEM59 protein includes an amino acid sequence as set forth in SEQ ID NO. 2.

In some embodiments, the TMEM59 protein and/or the functional fragment thereof includes an amino acid sequence as set forth in SEQ ID NO. 3.

In some embodiments, the immune cell includes a chimeric antigen receptor (CAR) and/or a coding element thereof.

In some embodiments, the CAR includes a transmembrane region.

In some embodiments, the transmembrane region of the CAR is selected from: a transmembrane region of the TMEM59 protein and a transmembrane region of CD8.

In some embodiments, the transmembrane region of the CAR includes an amino acid sequence as set forth in any one of SEQ ID NOs. 1 and 37.

In some embodiments, the CAR includes a hinge region.

In some embodiments, the hinge region of the CAR is selected from: a hinge region of the TMEM59 protein and a hinge region of CD8.

In some embodiments, the hinge region of the CAR includes an amino acid sequence as set forth in any one of SEQ ID NOs. 2 and 38.

In some embodiments, the CAR includes an intracellular domain, the intracellular domain of the CAR includes a signaling domain and/or a costimulatory domain.

In some embodiments, the signaling domain includes a signaling domain of CD3zeta.

In some embodiments, the costimulatory domain includes a costimulatory domain of 4-1BB.

In some embodiments, the CAR includes a targeting moiety.

In some embodiments, the targeting moiety includes ScFv.

In some embodiments, the targeting moiety specifically binds to and/or recognizes a tumor antigen.

In some embodiments, the targeting moiety specifically binds to and/or recognizes a target selected from a group consisting of: CD19, CD22 and GPC3.

In some embodiments, the immune cell includes the CAR as well as the TMEM59 protein and/or the functional fragment thereof, and wherein the CAR does not include the TMEM59 protein and/or the functional fragment thereof.

In some embodiments, the CAR includes the targeting moiety, the hinge region of CD8, the transmembrane region of CD8, the signaling domain of CD3zeta and the costimulatory domain of 4-1BB, wherein the TMEM59 protein and/or the functional fragment thereof include the transmembrane region and the hinge region of the TMEM59 protein.

In some embodiments, the CAR includes an amino acid sequence as set forth in any one of SEQ ID NOs. 49, 51, 55 and 56.

In some embodiments, the immune cell includes the CAR as well as the TMEM59 protein and/or the functional fragment thereof, and wherein said CAR includes the TMEM59 protein and/or the functional fragment thereof.

In some embodiments, the CAR includes the targeting moiety, the hinge region of the TMEM59 protein, the transmembrane region of the TMEM59 protein, the signaling domain of CD3zeta and the costimulatory domain of 4-1BB, wherein said TMEM59 protein and/or the functional fragment thereof includes the transmembrane region and the hinge region of the TMEM59 protein.

In some embodiments, the CAR includes an amino acid sequence as set forth in any one of SEQ ID NOs. 43, 57 and 44.

In some embodiments, the immune cell includes a T lymphocyte.

In another aspect, the present application provides a pharmaceutical composition, which includes the immune cell and optionally a pharmaceutically acceptable carrier.

In another aspect, the present application provides a use of the immune cell or the pharmaceutical composition in the preparation of a drug for treating tumors.

In some embodiments, the tumor includes a solid tumor and/or a non-solid tumor.

In some embodiments, the tumor includes B lymphocytic leukemia and/or liver cancer.

In another aspect, the present application provides a method for promoting the proliferation of an immune cell, which includes: allowing the immune cell to express a TMEM59 protein and/or a functional fragment thereof on its cell membrane, wherein the functional fragment includes at least a transmembrane region and a hinge region of the TMEM59 protein.

In another aspect, the present application provides a method for promoting the production of a memory immune cell, which includes: allowing the immune cell to express a TMEM59 protein and/or a functional fragment thereof on its cell membrane, wherein the functional fragment includes at least a transmembrane region and a hinge region of the TMEM59 protein.

In another aspect, the present application provides a method for enhancing the killing ability of an immune cell against a tumor, which includes: allowing the immune cell to express a TMEM59 protein and/or a functional fragment thereof on its cell membrane, wherein the functional fragment includes at least a transmembrane region and a hinge region of the TMEM59 protein.

In some embodiments, the expression includes allowing the immune cell to include a nucleic acid encoding the TMEM59 protein and/or the functional fragment thereof.

In some embodiments, the expression includes transfecting the immune cell with a plasmid including a nucleic acid encoding the TMEM59 protein and/or the functional fragment thereof.

In some embodiments, the immune cell includes a T lymphocyte.

In some embodiments, the immune cell includes a nucleic acid encoding the TMEM59 protein and/or the functional fragment thereof.

In some embodiments, the nucleic acid is in a viral vector.

In another aspect, the present application provides a method for inhibiting the proliferation of tumor cells and/or killing tumor cells, which includes: contacting the immune cell with the tumor cells.

In some embodiments, the method includes an in vitro method or an ex vivo method.

In some embodiments, the tumor includes a solid tumor and/or a non-solid tumor.

In some embodiments, the tumor includes B lymphocytic leukemia and/or liver cancer.

Those skilled in the art can easily perceive other aspects and advantages of the present application from the detailed description below. In the following detailed description, only exemplary embodiments of the present application are shown and described. As those skilled in the art will recognize, the content of the present application enables those skilled in the art to make changes to the disclosed specific embodiments without departing from the spirit and scope of the invention involved in the present application. Correspondingly, the drawings and descriptions in the specification of the present application are merely exemplary, rather than restrictive.

### BRIEF DESCRIPTION OF THE DRAWINGS

The specific features of the invention involved in the present application are shown in the appended claims. The characteristics and advantages of the invention involved in the present application can be better understood by referring to the exemplary embodiments and the accompanying drawings described in detail below. A brief description of the drawings is as follows:

FIG. 1 shows the number of memory cells in the H63 CAR-T lymphocyte;

FIG. 2 shows the number of memory cells in the H63 CAR-2A-TMEM59 T lymphocyte;

FIG. 3 shows the number of memory cells in the 21D4 CAR-2A-GFP T lymphocyte;

FIG. 4 shows the number of memory cells in the 21D4 CAR-2A-TMEM59 T lymphocyte;

FIG. 5 shows the proliferation number of the T lymphocyte of the present application;

FIG. 6 shows the killing rate of the T lymphocyte of the present application against a target cell;

FIG. 7 shows the trend of the biofluorescence intensity in tumor-bearing mice;

FIG. 8 shows the trend of the survival rate of tumor-bearing mice over time;

FIG. 9 shows an in vivo imaging map of the tumor-bearing mice.

### DETAILED DESCRIPTION OF THE EMBODIMENTS

The implementation of the present application will be illustrated in the following specific examples, and other advantages and effects of the present application will be easily known by those familiar with this technology from the content disclosed in the specification.

In the present application, the term “chimeric antigen receptor (CAR)” may refer to, for example, an artificial T cell receptor, a chimeric T cell receptor, or a chimeric immune receptor, and includes a genetically engineered receptor obtained by implanting artificial specificity onto a particular immune effector cell. The CAR can be used to confer the specificity of a monoclonal antibody to T cells, thus allowing the production of a large number of specific T cells, for example, so as to be used in the adoptive cellular immunotherapy. In some cases, the CAR can direct the cellular specificity to tumor-associated antigens. The CAR can include an intracellular activation domain, a transmembrane region, and an extracellular domain including a tumor-associated antigen binding region. For example, the CAR can include a fusion of a single-chain variable fragment (scFv) derived from a monoclonal antibody fused to the transmembrane region and the intracellular domain of CD3zeta. In some other cases, the CAR can also include additional costimulatory signaling domains, such as CD3zeta, FcR, CD27, CD28, CD137, DAP1O, and/or OX40. In some cases, the CAR can be co-expressed with a molecule, which may include a costimulatory molecule, a reporter gene for imaging (e.g., for positron emission tomography), a gene product that conditionally ablates T cells upon the addition of a prodrug, a homing receptor, a chemokine, a chemokine receptor, a cytokine, and a cytokine receptor.

In the present application, the term “antibody” generally refers to a protein or polypeptide sequence that specifically binds to an antigen, and that is derived from an immunoglobulin molecule. The antibody may be a polyclonal or monoclonal, a multi-chain or single-chain, or a complete immunoglobulin, and it can be derived from a natural source or a recombinant source. The antibody may be a tetramer of immunoglobulin molecules. The term “antibody fragment” generally refers to at least a part of a complete antibody or a recombinant variant thereof, and can also refer to an antigen-binding domain of a complete antibody, for example, an antigenic determining variable region, which is sufficient to confer recognition and specific binding of the antibody fragment to a target (e.g., an antigen). Examples of the antibody fragments include, but not limited to, antigen-binding fragments (Fab, Fab′, F(ab)₂, Fv fragments, F(ab′)₂, scFv, di-scFv and/or dAb), immunoconjugates, multi-specific antibodies (e.g., bispecific antibodies), antibody fragments, antibody derivatives, antibody analogues or fusion proteins, VHH domains, and multi-specific antibodies formed from the antibody fragments. The term “scFv” generally refers to a fusion protein including at least one antibody fragment containing a light chain variable region and at least one antibody fragment containing a heavy chain variable region, wherein the light chain variable region and the heavy chain variable region can be joined continuously by short flexible peptide linkers so as to be expressed as a single-chain polypeptide, and wherein the scFv maintains the specificity of the complete antibody from which it is derived. Unless otherwise specified, the scFv in the present application may have VL and VH variable regions in any order. For example, relative to the N-terminus and C-terminus of the polypeptide, scFv may include VL-linker-VH or may include VH-linker-VL. The part including an antibody or an antibody fragment thereof in the CAR of the present application may exist in various forms, including, e.g., single-domain antibody fragment (sdAb), single-chain antibody (scFv) and humanized antibody (Harlow et, al, 1999, In: Using Antibodies: A Laboratory Manual, Cold Spring Harbor Laboratory Press, NY; Harlow et, al, 1989, In: Antibodies: A Laboratory Manual, Cold Spring Harbor, N.Y.; Houston et, al, 1988, Proc. Natl. Acad. Sci. USA 85:5879-5883; Bird et, al, 1988, Science 242:423-426), wherein the antigen-binding domain is expressed as a part of a continuous polypeptide chain. In some cases, the antigen-binding domain of CAR of the present application includes an antibody fragment. In some other cases, CAR may include an antibody fragment containing scFv.

In the present application, the term “scFv” generally refers to a fusion protein including at least one antibody fragment containing a light chain variable region and at least one antibody fragment containing a heavy chain variable region, wherein the light chain variable region and the heavy chain variable region are contiguous (for example, via synthetic linkers, e.g., short flexible polypeptide linkers), and can be expressed in a form of a single-chain polypeptide, and wherein the scFv maintains the specificity of the complete antibody from which it is derived. In the present application, the scFv may have the VL and VH variable regions in any order (for example, relative to the N-terminus and C-terminus of the polypeptide). The scFv molecule has a general structure of NH₂-VL-linker-VH-COOH or NH₂-VH-linker-VL-COOH.

In the present application, the term “TMEM59” generally refers to the transmembrane protein 59, its functional variant and/or its functional fragment. The sequence of TMEM59 is known in the art. For example, the nucleotide sequence encoding the human TMEM59 protein and/or functional fragment thereof of the present application may include the nucleotide sequence of SEQ ID NO. 4. The human TMEM59 protein and/or functional fragment thereof of the present application may include the amino acid sequence of SEQ ID NO. 3.

In the present application, the term “co-stimulatory molecules” generally refers to correlation binding partner on the T cells, which specifically bind to co-stimulatory ligands, thereby mediating the co-stimulatory response of T cells, for example, but not limited to, proliferation. The co-stimulatory molecules are cell surface molecules or their ligands that are required for an effective immune response and are not antigenic receptors. The co-stimulatory molecules include, but not limited to, MHC class I molecules, BTLA and Toll-ligand receptors, as well as OX40, CD2, CD27, CD28, CDS, ICAM-1, LFA-I (CD18) and 4-1BB (CD137).

In the present application, the term “costimulatory domain” generally refers to any sequences of 4-1BB (CD137), which include, for example, stimulatory signaling domains of 4-1BB. They may include homologues, variants, isomers or functional fragments of 4-1BB.

In the present application, the term “signaling domain” refers to functional parts of a protein. The signaling domains can produce signals that promote the immune effector functions of CAR-containing cells, e.g., CAR-expressing cells, such as T cells or NK cells. For example, in CAR-expressing cells, examples of immune effector functions may include cytolytic activity and auxiliary activity, where the latter may include the secretion of cytokines. In some cases, signaling domains transduce effector functional signals and direct cells to perform specialized functions. Although the whole intracellular signaling domain can be used, in many instances, it is not necessary to use the intact chain of the whole domain. To the extent of using the truncated portions of the intracellular signaling domain, such truncated portions can be used in place of the intact chains as long as they transduce the effector functional signals. Therefore, the term “signaling domain” may include any truncated portions of the signaling domain that are sufficient to transduce the required functional signals.

In the present application, the term “transmembrane region” generally refers to a domain of a peptide, a polypeptide or a protein capable of spanning the cytoplasmic membrane. These domains can be used to anchor an antibody or its fragments on the cell membrane.

In the present application, the term “encoding” generally refers to the inherent property of a particular sequence of nucleotides in a polynucleotide such as a gene, cDNA or mRNA to act as a template for the synthesis of other multimers and macromolecules in a biological process, said multimers and macromolecules having either a defined sequence of nucleotides (i.e., rRNA, tRNA and mRNA) or a defined sequence of amino acids and the biological properties arising therefrom. Thus, if transcription and translation of an mRNA corresponding to a gene produces a protein in a cell or other biological system, the gene encodes the protein. Both the coding strand whose nucleotide sequence is identical to the mRNA sequence and is usually provided in the sequence listing, and the non-coding strand used as a template for the transcription of a gene or cDNA may be referred to as the protein or other product encoding the gene or cDNA. In the present application, the term “coding element” generally refers to a nucleic acid (an RNA or DNA molecule) including a nucleotide sequence encoding a protein (e.g., a chimeric antigen receptor (CAR)).

In the present application, the term “tumor antigen” generally refers to any molecules expressed on tumor cells (or related to the development of tumor cells) that are known or believed to have a role in the tumorigenic properties of tumor cells. These antigens generally have extracellular parts which can be present on the cell surface, and also have transmembrane parts and cytoplasmic parts that are joined together with the extracellular parts. These antigens can sometimes be present only on the surface of tumor cells but not on the surface of normal cells. Tumor antigens can be expressed specifically on tumor cells, or have tumor-specific mutations compared to normal cells. In such cases, they are referred to as tumor-specific antigens (TSAs). TSAs are unique to tumor cells, and do not occur on other cells of the body. Tumor antigens can be expressed not only in tumor cells, and can also be expressed on normal cells in conditions that cannot induce an immune tolerance state to the antigens, where they are referred to as tumor-associated antigens (TAAs). Compared to normal cells, TAAs can be overexpressed on tumor cells, or are prone to bind to antibodies in tumor cells due to the less compact structure of tumor tissues compared to normal tissues.

In the present application, the term “specifically bind to” generally refers to an antibody or ligand that recognizes and binds to a homologous binding ligand protein present in a sample, but substantially does not recognize and bind to other molecules in the sample.

In the present application, the term “CD19” generally refers to Cluster of Differentiation 19 protein, an antigenic determinant that can be detected in precursor cells of leukemia. The amino acid and nucleic acid sequences of human and mouse CD19 can be found in public databases, e.g., GenBank, UniProt and Swiss-Prot. For example, the amino acid sequence of human CD19 can be found in UniProt/Swiss-Prot Accession No. P15391, and the nucleotide sequence encoding human CD19 can be found in NCBI Accession No. NM_001178098. CD19 is expressed on most B-lineage cancers, which is also an early marker of B-cell progenitor cells. In the present application, “CD19” may include proteins containing mutations, for example, point mutations, fragments, insertions, deletions and spliced variants of full-length wild-type CD19.

In the present application, the term “does not include” generally refers to the exclusion of the possibility of a certain behavior, structure or structure. For example, “A does not include B” generally means to exclude the possibility of B occurring in A.

In the present application, the terms “peptide”, “polypeptide” and “protein” can be used interchangeably and generally refer to compounds composed of amino acid residues covalently linked by peptide bonds. The protein or peptide must contain at least two amino acids, and there is no limitation on the maximum number of amino acids that can be included in the protein or peptide sequence. The polypeptide may include any peptides or proteins that contain two or more amino acids linked to each other through peptide bonds. In the present application, this term refers to two short chains, which are also commonly known as peptides, oligopeptides and oligomers in the art, for example longs chains, which are commonly known as proteins in the art, of which there are many types. “Polypeptides” include, for example, bioactive fragments, substantially homologous polypeptides, oligopeptides, homodimers, heterodimers, variants of polypeptides, modified polypeptides, derivatives, analogues, fusion proteins, etc. Polypeptides include native peptides, recombinant peptides or combinations thereof.

In addition to particular proteins and nucleotides mentioned herein, the present application may also include their functional variants, derivatives, analogues, homologues and fragments thereof.

The term “functional variant” refers to a polypeptide having substantially the same amino acid sequence or encoded by substantially the same nucleotide sequence as the naturally occurring sequence and capable of having one or more activities of the naturally occurring sequence. In the context of the present application, the variant of any given sequence refers to a sequence in which a particular sequence of residues (either amino acid or nucleotide residues) has been modified so that the polypeptide or polynucleotide remains substantially at least one endogenous function. The variant sequences can be obtained through the addition, deletion, substitution, modification, replacement and/or variation of at least one amino acid residue and/or nucleotide residue present in a naturally occurring protein and/or polynucleotide, as long as the original functional activity is maintained. In the present application, the term “derivative” generally refers to a polypeptide or polynucleotide of the present application including any substitution, variation, modification, replacement, deletion and/or addition from/to one (or more) amino acid residues of the sequence, provided that the resulting polypeptide or polynucleotide substantially maintains at least one of its endogenous functions.

In the present application, the term “analogue” generally, with respect to a polypeptide or polynucleotide, includes any mimetic of the polypeptide or polynucleotide, that is, a chemical compound having at least one endogenous function of the polypeptide or polynucleotide that the mimetic mimics. In general, amino acids can be substituted, for example, at least 1 (e.g., 1, 2, 3, 4, 5, 6, 7, 8, 9, 10 or 20 or above) amino acids can be substituted, provided that the modified sequence substantially maintains the required activity or capability. Amino acid substitution may include the use of non-naturally occurring analogues. The protein or polypeptide used in the present application may also have deletion, insertion or substitution of amino acid residues, where the amino acid residues undergo silent changes and result in functionally equivalent proteins. Intentional amino acid substitutions can be made based on the similarity of the polarity, charge, solubility, hydrophobicity, hydrophilicity, and/or the amphoteric properties of the residues, as long as the endogenous function is retained. For example, negatively charged amino acids include aspartic acid and glutamic acid; positively charged amino acids include lysine and arginine; and amino acids containing uncharged polar head-groups with a similar hydrophilic value include asparagine, glutamine, serine, threonine and tyrosine.

In the present application, the term “homologue” generally refers to an amino acid sequence or a nucleotide sequence having a certain homology with a wild-type amino acid sequence and a wild-type nucleotide sequence. The term “homology” may be equivalent to the “identity” of sequences. Homologous sequences may include amino acid sequences that are at least 80%, 85%, 90%, 99.1%, 99.2%, 99.3%, 99.4%, 99.5%, 99.6%, 99.7%, 99.8% or 99.9% the same as the subject sequence. In general, homologues will contain the same active sites as the subject amino acid sequence, and the like. Homology may be considered on the basis of similarity (i.e., amino acid residues having similar chemical properties/functions), or homology can be expressed in terms of the sequence identity. In the present application, a sequence having a percentage identity in either of the SEQ ID NOs of the mentioned amino acid sequence or nucleotide sequence refers to a sequence having the percentage identity over the whole length of the mentioned SEQ ID NOs. In order to determine the sequence identity, alignment of sequences can be performed by a variety of ways known to those skilled in the art, for example, by using BLAST, BLAST-2, ALIGN, NEEDLE or Megalign (DNASTAR) software, etc. The persons skilled in the art are able to determine the suitable parameters suitable for alignment, including any algorithms required to achieve an optimal alignment in the full-length sequence being compared.

In the present application, the term “vector” generally refers to a vector containing a recombinant polynucleotide, where the recombinant polynucleotide includes an expression control sequence efficiently linked to a nucleotide sequence to be expressed. The vector includes cis-acting elements sufficient for expression; other elements for expression may be provided by the host cell or may be provided in an in-vitro expression system. The vector may include all expression vectors known in the art that can be incorporated into the recombinant polynucleotide, including cosmid, plasmid (e.g., naked or encapsulated in liposomes) and viruses (e.g., lentiviruses, retroviruses, adenoviruses, and adeno-associated viruses). The term “lentivirus” refers to a genus of Lentiviridae. Lentiviruses are unique in retroviruses and capable of infecting non-dividing cells; they can deliver a significant amount of genetic information into DNA of host cells, therefore, they are one of the most efficient gene delivery vector methods. HIV, SIV and FIV are all examples of lentiviruses. The term “lentiviral vector” generally refers to vectors derived from at least one part of a lentivirus genome, including particularly those self-inactivated lentiviral vectors provided in Milone et al., Mol. Ther. 17(8):1453-1464 (2009). Other examples of lentiviral vectors that can be used clinically include, but not limited to, e.g., LENTIVECTOR® gene delivery technology of Oxford BioMedica, LENTIMAX™ vector system of Lentigen, etc. Non-clinical lentiviral vectors are also available and are known to those skilled in the art.

In the present application, the term “subject” generally refers to living organisms that can trigger immune responses (e.g., mammals, human).

In the present application, the term “treat” generally refers to slowing or improving the progression, severity, and/or duration of a proliferative condition, or improving one or more symptoms (e.g., one or more distinguishable symptoms) of a proliferative condition as a result of the administration of one or more therapies (for example, one or more therapeutic agents, such as the CAR of the present application). In the present application, the term “treat” may also refer to improving at least one measurable physical parameter of a proliferative condition, such as tumor growth, which physical parameter does not have to be identifiable to the patient. The term “treatment” in the present application may also refer to inhibiting the progression of a proliferative condition by for example stabilizing discernible symptoms in a physical manner, by for example stabilizing physical parameters in a physiological manner, or both. In some cases, the term “treatment” may refer to reducing or stabilizing the tumor size or the count of cancer cells.

In the present application, the term “include” generally refers to the inclusion of explicitly specified features, but not excluding other elements.

In the present application, the term “about” generally refers to varying in a range of 0.5%-10% above or below a specified value, for example, varying in a range of 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, or 10% above or below a specified value.

In one aspect, the present application provides an immune cell, which may include a chimeric antigen receptor (CAR) and/or a coding element thereof.

### Targeting Moiety

The CAR of the immune cell of the present application may include a targeting moiety. The targeting moiety may be an antigen-binding domain, and the selection of the targeting moiety depends on the type and number of ligands defining the surface of the target cells. In one aspect, CAR-mediated T cell responses can be directed to target antigens by engineering an antigen-binding domain that specifically binds the desired antigens into the CAR. In some cases, the targeting moiety may specifically bind to and/or recognize tumor antigens. The tumor antigens may be tumor specific antigens (TSAs) or tumor-associated antigens (TAAs). According to the desired tumor antigen to be targeted, the CAR of the present application can be engineered to include an appropriate antigen-binding portion specific to the desired antigen. The targeting moiety may be: including, but not limited to, monoclonal antibodies, polyclonal antibodies, recombinant antibodies, human antibodies, humanized antibodies and functional fragments thereof, including, but not limited to, single-domain antibodies, such as heavy chain variable domains (VH), light chain variable domains (VL), and variable domains (VHH) of camelid-derived nanobodies. In some cases, the targeting moiety of the present application may be antibody single-chain variable regions (scFv). Generally, for ease of practical applications, the targeting moiety of CAR may include human or humanized residues for the antigen-binding domain of an antibody or an antibody fragment.

In some cases, the targeting moiety of the CAR of the present application may target a CD19 target. For example, the targeting moiety of the CAR of the present application may be the single-chain variable region of a humanized CD19 monoclonal antibody. For example, the single-chain variable region of the humanized CD19 monoclonal antibody may include an amino acid sequence as set forth in SEQ ID NO. 41. Further for example, the single-chain variable region of the humanized CD19 monoclonal antibody can be encoded by a nucleotide sequence as set forth in SEQ ID NO. 11. Alternatively, the targeting moiety may also be the single-chain variable region of a full humanized CD19 monoclonal antibody. For example, the single-chain variable region of the full humanized CD19 monoclonal antibody may include an amino acid sequence as set forth in SEQ ID NO. 42. Further for example, the single-chain variable region of the full humanized CD19 monoclonal antibody can be encoded by a nucleotide sequence as set forth in SEQ ID NO. 12.

In some other cases, the targeting moiety of the CAR of the present application may target a CD22 target. For example, the targeting moiety targeting CD22 can be encoded by an amino acid sequence as set forth in SEQ ID NO. 48. For example, the targeting moiety targeting CD22 can be encoded by a nucleotide sequence as set forth in SEQ ID NO. 35.

In some other cases, the targeting moiety of the CAR of the present application may target a GPC3 target. For example, the targeting moiety targeting GPC3 may include a nucleotide sequence as set forth in SEQ ID NO. 54. For example, the targeting moiety targeting GPC3 may include a nucleotide sequence as set forth in SEQ ID NO. 36.

In the present application, the targeting moiety of the CAR may specifically bind to and/or recognize a target selected from a group consisting of: CD19, CD22 and GPC3.

The targeting moiety of the CAR of the present application may specifically bind to and/or recognize the targeting moieties of one or more (for example, two or more) targets. In some cases, the CAR of the present application may include a targeting moiety with binding and/or recognition specificity for one target, where the target may be selected from a group consisting of: CD19, CD22 and GPC3. In some other cases, the targeting moiety of the CAR of the present application may include a first targeting moiety with binding and/or recognition specificity for a first target, and a second targeting moiety with binding and/or recognition specificity for a second target, where the target is selected from a group consisting of: CD19 and CD22, CD19 and GPC3 as well as CD22 and GPC3.

### Transmembrane Region

The CAR of the immune cell of the present application may include a transmembrane region. In some cases, the hinge region may be a transmembrane region naturally associated with one domain in the CAR. In some other cases, the transmembrane region can be modified by amino acids to avoid the binding of such a domain with transmembrane domains of the same or different surface membrane proteins, thereby minimizing the interaction with other members of the receptor complex. The transmembrane domains may be naturally-derived or synthetically-derived. In the case of naturally-derived, the domains may be derived from any membrane binding or transmembrane proteins. The transmembrane domains for particular uses in the present application may be or may be derived from those feasible in the art. The transmembrane domains may also be synthesized, which, in such cases, may mainly include primarily hydrophobic amino acid residues. In some cases, the transmembrane regions may be a transmembrane region of a TMEM59 protein. For example, the transmembrane region of the TMEM59 protein may include an amino acid sequence as set forth in SEQ ID NO. 1. Further for example, the transmembrane region of the TMEM59 protein can be encoded by a nucleotide sequence as set forth in SEQ ID NO. 5. In some other cases, the transmembrane region may be a transmembrane region of CD8. For example, the transmembrane region of CD8 may include an amino acid sequence as set forth in SEQ ID NO. 37. Further for example, the transmembrane region of CD8 can be encoded by a nucleotide sequence as set forth in SEQ ID NO. 34.

### Hinge Region

In some cases, the transmembrane region can be linked to an extracellular region of CAR through a hinge, for example, the targeting moiety of the CAR. The CAR of the immune cell of the present application may include a hinge region. In some cases, the hinge region may be a hinge region of a TMEM59 protein. For example, the hinge region of the TMEM59 protein may include an amino acid sequence as set forth in SEQ ID NO. 2. Further for example, the TMEM59 protein can be encoded by a nucleotide sequence as set forth in SEQ ID NO. 6. In some other cases, the hinge region may be a hinge region of CD8. For example, the hinge region of CD8 may include an amino acid sequence as set forth in SEQ ID NO. 38. Further for example, the hinge region of CD8 can be encoded by a nucleotide sequence as set forth in SEQ ID NO. 8.

### Intracellular Domain

In the present application, the CAR of the immune cell may also include an intracellular domain. The intracellular domain may include a signaling domain. Examples of intracellular signaling domains that can be used in the CAR of the present application may generally include cytoplasmic sequences of T cell receptors (TCRs) and co-receptors that act together in order to trigger signal transduction after the adaption of antigen receptors, as well as any derivatives or variants of these sequences and any one recombinant sequence with the same functional capacity.

It is known that signals generated through the TCRs alone are insufficient to completely activate T cells, and secondary or co-stimulatory signals are also required. Therefore, the activation of T cells can be considered to be mediated by two different kinds of cytoplasmic signaling sequences: antigen-dependent primarily activated signaling sequences initiated with TCR (primary cytoplasmic signaling sequences) and signaling sequences providing secondary or co-stimulatory signals by acting in an antigen-independent manner (secondary cytoplasmic signaling sequences, e.g., costimulatory domains).

Primary cytoplasmic signaling sequences regulate the primary activation of TCR complexes in a stimulatory manner or in an inhibitory manner. The primary cytoplasmic signaling sequences that act in a stimulatory manner may include signaling motifs, e.g., immune receptor tyrosine-based activation motifs (ITAMs). The ITAMs that can be used as the primary cytoplasmic signaling sequences in the present application may include any signaling domains feasible in the art. In some cases, the signaling domains of the present application may be a signaling domain of CD3zeta, which may include a nucleotide sequence as set forth in SEQ ID NO. 9.

In some cases, the intracellular domain of the CAR of the present application may include a signaling domain of CD3zeta. Alternatively, the signaling domain can also combine with any other feasible one or more intracellular domains if it is useful for the functioning of the CAR of the present application. For example, the intracellular domain of the CAR may include the chain portion and the costimulatory domain of CD3zeta. The costimulatory domain generally refers to the portion in the CAR which includes the intracellular domain of the co-stimulatory molecule. The co-stimulatory molecule generally refers to cell surface molecules other than antigen receptors or their ligands that are required for the effective response of lymphocytes to an antigen. Examples of co-stimulatory molecules suitable for the present application may include those feasible in the art.

In some cases, the costimulatory domain of the present application may include a costimulatory domain of 4-1BB. For example, the costimulatory domain of 4-1BB may include a nucleotide sequence as set forth in SEQ ID NO. 10.

TMEM59 Protein and/or Functional Fragment Thereof

The immune cell of the present application may include a TMEM59 protein and/or a functional fragment thereof located on the cell membrane.

In some cases, the immune cell of the present application may include a transmembrane region and a hinge region of the TMEM59 protein. In some cases, the TMEM59 protein may be a human TMEM59 protein. For example, the transmembrane region of the TMEM59 protein may include an amino acid sequence as set forth in SEQ ID NO. 1. For example, the hinge region of the TMEM59 protein may include an amino acid sequence as set forth in SEQ ID NO. 2.

In some cases, the immune cell may include a TMEM59 protein and/or a functional fragment thereof, wherein the functional fragment includes at least a transmembrane region and a hinge region of the TMEM59 protein. In some cases, the functional fragment may be a human TMEM59 protein and/or a functional fragment thereof. For example, the transmembrane region of the TMEM59 protein may include an amino acid sequence as set forth in SEQ ID NO. 1. For example, the hinge region of the TMEM59 protein may include an amino acid sequence as set forth in SEQ ID NO. 2. For example, the TMEM59 protein and/or the functional fragment thereof may include an amino acid sequence as set forth in SEQ ID NO. 3.

In some cases, the TMEM59 protein and/or the functional fragment thereof can be located on the cell membrane of the immune cell. In some other cases, the immune cell can be modified to include the TMEM59 protein and/or the functional fragment thereof on the cell membrane.

The CAR of the present application may also include a signal peptide. In some cases, the signal peptide may include a CD8 signal peptide. For example, the CD8 signal peptide may include an amino acid sequence as set forth in SEQ ID NO. 46, for example, the CD8 signal peptide can be encoded by a nucleotide sequence as set forth in SEQ ID NO. 16.

The immune cell of the present application may include the CAR as well as the TMEM59 protein and/or the functional fragment thereof. In some cases, the TMEM59 protein and/or the functional fragment thereof can be located on the cell membrane of the immune cell.

### TMEM59 Co-Expressed by Chimeric Antigen Receptors

In some cases, the CAR may not include the TMEM59 protein and/or the functional fragment thereof. In some specific cases, the CAR may include the targeting moiety, the hinge region, the transmembrane region, the signaling domain and the costimulatory domain. The CAR may include the targeting moiety, the hinge region of CD8, the transmembrane region of CD8, the signaling domain of CD3zeta and the costimulatory domain of 4-1BB. In some cases, the CAR can co-express the TMEM59 protein and/or the functional fragment thereof. In some cases, the intracellular domain of the CAR can be directly or indirectly linked to the TMEM59 protein and/or the functional fragment thereof. For example, the CAR may include an amino acid sequence as set forth in any one of SEQ ID NOs. 49, 51, 55 and 56. For example, the CAR can be encoded by a nucleotide sequence as set forth in any one of SEQ ID NOs. 19, 21, 25 and 28. For example, the TMEM59 protein and/or the functional fragment thereof may include an amino acid sequence as set forth in any one of SEQ ID NOs. 1-3. For example, the TMEM59 protein and/or the functional fragment thereof can be encoded by a nucleotide sequence as set forth in any one of SEQ ID NOs. 4-6.

For example, the CAR may include the targeting moiety of the single-chain variable region of the humanized CD19 monoclonal antibody, the hinge region of CD8, the transmembrane region of CD8, the signaling domain of CD3zeta and the costimulatory domain of 4-1BB. For example, the CAR may include an amino acid sequence as set forth in SEQ ID NO. 49. Further for example, the CAR can be encoded by a nucleotide sequence as set forth in SEQ ID NO. 19. In some cases, the CAR can co-express the TMEM59 protein and/or the functional fragment thereof. For example, the TMEM59 protein and/or the functional fragment thereof may include an amino acid sequence as set forth in SEQ ID NO. 3. Further for example, the TMEM59 protein and/or the functional fragment thereof can be encoded by an amino acid sequence as set forth in SEQ ID NO. 4. The protein in the co-expression of the TMEM59 protein and/or the functional fragment thereof by the CAR of the present application may include an amino acid sequence as set forth in SEQ ID NO. 50 or 52. Further for example, the protein in the co-expression of the TMEM59 protein and/or the functional fragment thereof by the CAR of the present application can be encoded by a nucleotide sequence as set forth in any one of SEQ ID NOs. 20, 32, 36 and 29.

For example, the CAR may include the targeting moiety of the single-chain variable region of the full humanized CD19 monoclonal antibody, the hinge region of CD8, the transmembrane region of CD8, the signaling domain of CD3zeta and the costimulatory domain of 4-1BB. For example, the CAR may include an amino acid sequence as set forth in SEQ ID NO. 51. For example, the CAR can be encoded by a nucleotide sequence as set forth in SEQ ID NO. 21. In some cases, the CAR can co-express the TMEM59 protein and/or the functional fragment thereof. For example, the TMEM59 protein and/or the functional fragment thereof may include a nucleotide sequence as set forth in SEQ ID NO. 3. For example, the TMEM59 protein and/or the functional fragment thereof can be encoded by a nucleotide sequence as set forth in SEQ ID NO. 4.

For example, the CAR may include the GPC3 targeting moiety, the hinge region of CD8, the transmembrane region of CD8, the signaling domain of CD3zeta and the costimulatory domain of 4-1BB. For example, the CAR may include an amino acid sequence as set forth in SEQ ID NO. 55. For example, the CAR can be encoded by a nucleotide sequence as set forth in SEQ ID NO. 25. In some cases, the CAR can co-express the TMEM59 protein and/or the functional fragment thereof. For example, the TMEM59 protein and/or the functional fragment thereof may include a nucleotide sequence as set forth in SEQ ID NO. 3. For example, the TMEM59 protein and/or the functional fragment thereof can be encoded by a nucleotide sequence as set forth in SEQ ID NO. 4. For example, the CAR may include CD19 and CD22 targeting moieties, the hinge region of CD8, the transmembrane region of CD8, the signaling domain of CD3zeta and the costimulatory domain of 4-1BB. For example, the CAR may include a nucleotide sequence as set forth in SEQ ID NO.28. In some cases, the CAR can co-express the TMEM59 protein and/or the functional fragment thereof. For example, the TMEM59 protein and/or the functional fragment thereof may include an amino acid sequence as set forth in SEQ ID NO. 3. For example, the TMEM59 protein and/or the functional fragment thereof can be encoded by a nucleotide sequence as set forth in SEQ ID NO. 4.

In the present application, the CAR and the TMEM59 protein and/or the functional fragment thereof can be co-expressed through a shear peptide. For example, the shear peptide may be a 2A sequence. Further for example, the 2A sequence may include an amino acid sequence as set forth in SEQ ID NO. 47. Further for example, the 2A sequence can be encoded by a nucleotide sequence as set forth in SEQ ID NO. 17. In the present application, the CAR and the TMEM59 protein and/or the functional fragment thereof can be co-expressed through an IRES sequence. Further for example, the IRES sequence may include a nucleotide sequence as set forth in SEQ ID NO.18.

### TMEM59 Modified Chimeric Antigen Receptors

In some other cases, the CAR may include the TMEM59 protein and/or the functional fragment thereof. In some specific cases, the CAR may include the targeting moiety, the hinge region, the transmembrane region, the signaling domain and the costimulatory domain. In some cases, the CAR may include the targeting moiety, the hinge region of the TMEM59 protein, the transmembrane region of the TMEM59 protein, the signaling domain and the costimulatory domain. For example, the CAR may include the targeting moiety, the hinge region of the TMEM59 protein, the transmembrane region of the TMEM59 protein, the signaling domain of CD3zeta and the costimulatory domain of 4-1BB.

For example, the CAR may include an amino acid sequence as set forth in any one of SEQ ID NOs. 43, 57 and 44. For example. The CAR can be encoded by a nucleotide sequence as set forth in any one of SEQ ID NOs. 13, 14, 31, 32 and 33.

For example, the CAR may include the targeting moiety of the single-chain variable region of the full humanized CD19 monoclonal antibody, the hinge region of the TMEM59 protein, the transmembrane region of the TMEM59 protein, the signaling domain of CD3zeta and the costimulatory domain of 4-1BB. For example, the CAR may include an amino acid sequence as set forth in SEQ ID NO. 44. For example, the CAR can be encoded by a nucleotide sequence as set forth in SEQ ID NO. 7 or 33.

For example, the CAR may include CD19 and CD22 targeting moieties, the hinge region of the TMEM59 protein, the transmembrane region of the TMEM59 protein, the signaling domain of CD3zeta and the costimulatory domain of 4-1BB. For example, the CAR may include an amino acid sequence as set forth in SEQ ID NO. 57. For example, the CAR can be encoded by a nucleotide sequence as set forth in SEQ ID NO. 31 or 32.

For example, the CAR may include GPC3 targeting moiety, the hinge region of the TMEM59 protein, the transmembrane region of the TMEM59 protein, the signaling domain of CD3zeta and the costimulatory domain of 4-1BB. For example, the CAR may include a nucleotide sequence as set forth in SEQ ID NO. 43. For example, the CAR can be encoded by a nucleotide sequence as set forth in SEQ ID NO. 13 or 14.

### Vectors, Cells and Preparation Methods

In one aspect, the present application provides one or more isolated nucleic acids, which can encode the CAR polypeptide or portions thereof. Generally, the expression of a natural or synthetic nucleic acid encoding a CAR is achieved by operatively linking a nucleic acid encoding a CAR polypeptide or a portion thereof to a promoter and incorporating the construct into an expression vector. The vector may be suitable for replicating and integrating eukaryotic cells. Typical cloning vectors include transcriptional and translational terminators, initiation sequences and promoters that can be used for regulating the expression of desired nucleic acid sequences.

The nucleic acid sequences encoding the desired molecules can be obtained by using recombinant methods known in the art, such as, for example using standard techniques, by screening libraries from cells expressing the gene, or by obtaining the gene from vectors known to include the gene, or by direct isolation from cells and tissues including the gene. Nucleic acids can be cloned into the vectors, for example, nucleic acids can be cloned into vectors including, but not limited to, plasmids, phagemids, phage derivatives, animal viruses and cosmids. In particular, the target vectors may include expression vectors, replication vectors, probe-producing vectors, and sequencing vectors.

In another aspect, the present application provides cells including the nucleic acids or vectors of the present application. In some cases, the cells may be in vitro cells, which includes nucleic acids encoding the CAR of the present application. In other cases, the cells of the present application, e.g., in vitro cells, may include polypeptides encoded by the CAR of the present application.

For the nucleic acids, vectors or polypeptides of the present application, any cells can be used as host cells. In some embodiments, cells may be prokaryotic cells, fungal cells, yeast cells or higher eukaryotic cells, e.g., mammal cells. Suitable prokaryotic cells include, but not limited to, eubacteria, e.g., Gram-negative or Gram-positive organisms. In some cases, cells may be human cells. In some other cases, cells may be immune cells. The immune cells of the present application are selected from a group consisting of: T lymphocytes, B lymphocytes, tumor infiltrating lymphocytes (TILs), TCR expression cells, natural killer (NK) cells, dendritic cells, granulocytes, innate lymphoid cells, megakaryocytes, monocytes, macrophages, platelets, thymocytes and myeloid cells. In some cases, the immune cells may include T lymphocytes. In some other cases, T lymphocytes may be tumor infiltrating lymphocytes (TILs), autologous T lymphocytes, engineered autologous T lymphocytes (eACTTMs), allogeneic T cells, heterologous T cells, or any combinations thereof. In some cases, T lymphocytes can be obtained from donor subjects. The cells of the present application can be obtained from any sources known in the art. For example, T lymphocytes can be differentiated from hematopoietic stem cell populations in vitro, or T lymphocytes can be obtained from subjects. T lymphocytes can be obtained from, e.g., peripheral blood mononuclear cells, bone marrow, lymph node tissues, umbilical cord blood, thymus tissues, tissues from infection sites, ascitic fluid, pleural effusion, spleen tissues as well as tumors. In addition, T lymphocytes can be derived from one or more T lymphocyte lines available in the art. T cells can also be obtained from blood samples collected from subjects by any number of technologies (e.g., FICOLL™ isolation and/or apheresis) known to a person skilled in the art.

Methods for introducing a gene into cells and expressing a gene into cells are known in the art. Any methods in the art can be used to introduce a vector into host cells, e.g., mammalian, bacterial, yeast or insect cells. For example, expression vectors can be transferred into the host cells by physical, chemical or biological means.

Physical methods for introducing nucleic acids into host cells include calcium phosphate precipitation, liposomal transfection, particle bombardment, microinjection, electroporation, etc. The method for producing cells including vectors and/or exogenous nucleic acids are well known in the art (see, for example, Sambrook et al., 2001, Molecular Cloning: A Laboratory Manual, Cold Spring HarborLaboratory, New York and other manuals of Virology and Molecular Biology). Chemical means for introducing nucleic acids into host cells include colloidal dispersion systems, such as macromolecular complexes, nanocapsules, microspheres, beads; and lipid-based systems, including oil-in-water emulsions, micelles, mixed micelles and liposomes. Biological methods for introducing nucleic acids of interest into host cells include the use of DNA and RNA vectors. Currently widely used methods for inserting a gene into mammals (e.g., human cells) can utilize viral vectors. The viruses used as vectors include, but are not limited to, retroviruses, adenoviruses, adeno-associated viruses, herpes viruses, and lentiviruses. Generally, suitable vectors contain origins of replication, promoter sequences, convenient restriction endonuclease sites and one or more optional markers that act in at least one organism.

### Pharmaceutical Composition

The present application provides a pharmaceutical composition, which includes the immune cell and optionally a pharmaceutically acceptable carrier. In some cases, the pharmaceutical composition may include a pharmaceutically acceptable carrier, diluent, solubilizer, emulsifier, preservative and/or adjuvant. In some other cases, the pharmaceutical composition may include an excipient. In some other cases, the pharmaceutical composition may include a polynucleotide encoding the CAR of the present application. In some other cases, the pharmaceutical composition may include the CAR encoded by the polynucleotide of the present application. In some other cases, the composition may include T cells containing CAR.

The pharmaceutical composition of the present application can be prepared for parenteral delivery, for inhalation or for digestive tract delivery, e.g., oral administration. The preparation of such a pharmaceutically acceptable composition is within the capability of those skilled in the art. In some cases, a buffer solution is used to maintain the pharmaceutical composition at physiological pH or below, usually in a pH range of about 5 to about 8. In some cases, when considering parenteral administration, the pharmaceutical composition is in the form of an aqueous solution that is free of pyrogen and parenterally acceptable. In some cases, the pharmaceutical composition of the present application can be formulated into an appropriately preserved sterile isotonic solution. In some embodiments, preparations with desired molecules of polymeric compounds, beads or liposomes that provide controlled or sustained release of the product can be prepared and then delivered through depot injection. In some cases, the desired molecules can be imported by an implantable drug delivery device.

### Uses, Methods

In one aspect, the present application provides a method for promoting the proliferation of immune cells, which includes: allowing the immune cell to express a TMEM59 protein and/or a functional fragment thereof on its cell membrane, wherein the functional fragment includes at least a transmembrane region and a hinge region of the TMEM59 protein. In vitro amplification of CAR-T cells after antigen stimulation can be measured by flow cytometry, and the amplification of CAR-T cells maintained in the absence of restimulation can also be measured (see, for example, Milone et, al., Molecular Therapy 17 (8): 1453-1464 (2009)). In another aspect, immune cells can also be counted after culturing the immune cells under suitable conditions so that they can express the TMEM59 protein and/or the functional fragment thereof (for example, counting with a cell counter).

The immune cells of the present application may include T lymphocytes. For example, T cells may be tumor infiltrating lymphocytes (TILs), autologous T cells, engineered autologous T cells (eACTTMs), allogeneic T cells, heterologous T cells, or any combinations thereof. In some cases, T cells can be obtained from donor subjects. In some other cases, donor subjects may be human patients with cancers or tumors. In other cases, donor subjects may be human patients without cancers or tumors.

In another aspect, the present application provides a method for promoting the production of a memory immune cell. In some cases, the immune cells of the present application can undergo a stable process of in vivo T lymphocyte expansion, and can persist for an extended period of time. In some other cases, the immune cells of the present application may develop into memory immune cells that can be reactivated to inhibit any additional tumor forms or growth. For example, the immune cells of the present application can undergo strong in vivo T lymphocyte expansion to form specific memory T cells at a high level in blood and bone marrow, and persist for an extended period of time to prolong the survival time of chimeric antigen receptor T lymphocytes.

In another aspect, the present application provides a use of the immune cell or the pharmaceutical composition in the preparation of a drug for treating tumors. In some cases, the tumor may include a solid tumor and/or a non-solid tumor. The solid tumor is usually an abnormal mass of tissues that does not contain cysts or fluid areas. The solid tumor may be benign or malignant. The non-solid tumor may be, such as, a hematologic tumor. Hematologic cancers are cancers of blood or bone marrow, for example, leukemia and lymphoma. For example, the tumor may include B lymphocytic leukemia and/or liver cancer.

In another aspect, the present application further provides a method for enhancing the killing ability of an immune cell against a tumor, which includes: allowing the immune cell to express a TMEM59 protein and/or a functional fragment thereof on its cell membrane, wherein the functional fragment includes at least a transmembrane region and a hinge region of the TMEM59 protein. In some cases, the expression includes allowing the immune cell to include a nucleic acid encoding the TMEM59 protein and/or the functional fragment thereof, so as to transduce cells with the nucleic acid disclosed in the present application under suitable conditions. For example, a nucleic acid encoding CAR is used to transduce cells. Further for example, a vector including a nucleic acid encoding CAR is used to transduce cells.

The present application further provides a method for inhibiting the proliferation of tumor cells and/or killing tumor cells, which includes: contacting the immune cells with the tumor cells. Wherein, the method includes an in vitro method or an ex vivo method. In some cases, the immune cells can be contacted with the tumor cells at 1:500 to 500:1 or any number ratio therebetween. In some cases, the number ratio of the immune cells to the tumor cells may be in a range of 1:100 to 100:1 and any ratio therebetween. For example, it may be from 20:1 to 1:20.

Without wishing to be limited by any theory, the following examples are only intended to illustrate the fusion protein, the preparation methods and the uses of the present application, and are not intended to limit the inventive scope of the present application.

### EXAMPLES

### Example 1. Determination of Chimeric Antigen Receptor Sequences

H63 represents a single-chain variable region of a humanized CD19 monoclonal antibody, whose sequence structure is as set forth in SEQ ID NO: 11; 21D4 represents a single-chain variable region of a full humanized CD19 monoclonal antibody, whose sequence structure is as set forth in SEQ ID NO: 12; the sequence structure of the GPC3 targeting moiety is as set forth in SEQ ID NO: 36; the sequence structure of the CD22 targeting moiety is as set forth in SEQ ID NO: 35; the sequence structure of the green fluorescent protein (GFP) is as set forth in SEQ ID NO: 15.

1.1 Determination of Chimeric Antigen Receptor Co-Expressed TMEM59 Sequences

Structure of chimeric antigen receptor co-expressed TMEM59 sequences: CD8 signal peptide-tumor specific antigen-binding single-chain variable region-human CD8 hinge region-human CD8 transmembrane region-human 4-1BB costimulatory domain-CD3zeta signaling domain-IRES sequence or 2A sequence-TMEM59 protein full-length gene sequence. The CD8 signal peptide, human CD8 hinge region, human CD8 transmembrane region, human 4-1BB costimulatory domain, CD3zeta signaling domain and TMEM59 gene full-length sequence are searched from GenBank Database of the U.S. National Library of Medicine (https://www.ncbi.nlm.nih.gov/gene). Moreover, codons are optimized to facilitate the expression in human cells.

Where, the CD8 signal peptide may include a nucleotide sequence as set forth in SEQ ID NO: 16, the human CD8 hinge region may include a nucleotide sequence as set forth in SEQ ID NO: 8, the human CD8 transmembrane region may include a nucleotide sequence as set forth in SEQ ID NO: 7, the human 4-1BB costimulatory domain may include a nucleotide sequence as set forth in SEQ ID NO: 10, the CD3zeta signaling domain may include a nucleotide sequence as set forth in SEQ ID NO: 9, and the TMEM59 gene full-length sequence may include a nucleotide sequence as set forth in SEQ ID NO: 4.

The above gene sequences are ligated sequentially in a sequence of CD8 signal peptide, tumor specific antigen-binding single-chain variable region, human CD8 hinge region, human CD8 transmembrane region, human 4-1BB costimulatory domain, CD3zeta signaling domain, 2A (SEQ ID NO: 17) or IRES (SEQ ID NO: 18), TMEM59 protein full-length gene sequence, and ligated into a lentiviral vector (from Clontech 631988) through whole gene synthesis.

The structures of chimeric antigen receptor co-expressed TMEM59 sequences are as below,

Structure of H63 CAR-2A-TMEM59 sequence: CD8 signal peptide-H63-human CD8 hinge region-human CD8 transmembrane region-human 4-1BB costimulatory domain-CD3zeta signaling domain-2A-TMEM59 protein full-length gene sequence, and the nucleotide sequence is as set forth in SEQ ID NO: 20;

Structure of 21D4 CAR-2A-TMEM59 sequence: CD8 signal peptide-21D4-human CD8 hinge region-human CD8 transmembrane region-human 4-1BB costimulatory domain-CD3zeta signaling domain-2A-TMEM59 protein full-length gene sequence, and the nucleotide sequence is as set forth in SEQ ID NO: 22;

Structure of GPC3 CAR-IRES-TMEM59 sequence: CD8 signal peptide-GPC3-human CD8 hinge region-human CD8 transmembrane region-human 4-1BB costimulatory domain-CD3zeta signaling domain-IRES-TMEM59 protein full-length gene sequence, and the nucleotide sequence is as set forth in SEQ ID NO: 26;

Structure of CD19CD22 CAR-IRES-TMEM59 sequence: CD8 signal peptide-CD19CD22-human CD8 hinge region-human CD8 transmembrane region-human 4-1BB costimulatory domain-CD3zeta signaling domain-IRES-TMEM59 protein full-length gene sequence, and the nucleotide sequence is as set forth in SEQ ID NO: 29.

1.2 Determination of TMEM59 Modified Chimeric Antigen Receptor Sequence

Structure of TMEM59 modified chimeric antigen receptor sequence: CD8 signal peptide-tumor specific antigen-binding single-chain variable region-human TMEM59 hinge region-human TMEM59 transmembrane region-human 4-1BB costimulatory domain-CD3zeta signaling domain gene sequence. The CD8 signal peptide, human CD8 hinge region, human CD8 transmembrane region, human 4-1BB costimulatory domain, CD3zeta signaling domain and human TMEM59 hinge region, human TMEM59 transmembrane region sequence are searched from GenBank Database of the National Library of Medicine (https://www.ncbi.nlm.nih.gov/gene). Moreover, codons are optimized to facilitate the expression in human cells. The above gene sequences are ligated sequentially in a sequence of CD8 signal peptide, tumor specific antigen-binding single-chain variable region, human TMEM59 hinge region, human TMEM59 transmembrane region, human 4-1BB costimulatory domain, CD3zeta signaling domain, and ligated into a lentiviral vector (from Clontech) through whole gene synthesis.

Where, the human TMEM59 hinge region may include a nucleotide sequence as set forth in SEQ ID NO: 6, the human TMEM59 transmembrane region may include a nucleotide sequence as set forth in SEQ ID NO: 5. The structures of the TMEM59 modified chimeric antigen receptor sequences are as below,

Structure of 21D4-59TM CAR-IRES-GFP sequence: CD8 signal peptide-21D4-human TMEM59 hinge region-human TMEM59 transmembrane region-human 4-1BB costimulatory domain-CD3zeta signaling domain-IRES-GFP gene sequence, and the nucleotide sequence is as set forth in SEQ ID NO: 7;

Structure of GPC3-59TM CAR-IRES-GFP sequence: CD8 signal peptide-GPC3-human TMEM59 hinge region-human TMEM59 transmembrane region-human 4-1BB costimulatory domain-CD3zeta signaling domain-IRES-GFP gene sequence, and the nucleotide sequence is as set forth in SEQ ID NO: 14;

Structure of CD19CD22-59TM CAR-IRES-GFP sequence: CD8 signal peptide-CD19CD22-human TMEM59 hinge region-human TMEM59 transmembrane region-human 4-1BB costimulatory domain-CD3zeta signaling domain-IRES-GFP gene sequence, and the nucleotide sequence is as set forth in SEQ ID NO: 32.

Structure of CD19CD22-59TM CAR sequence: CD8 signal peptide-CD19CD22-human TMEM59 hinge region-human TMEM59 transmembrane region-human 4-1BB costimulatory domain-CD3zeta signaling domain, and the nucleotide sequence is as set forth in SEQ ID NO: 31.

1.3 Determination of Control Sequence

The following sequence structures are ligated into a lentiviral vector (from Clontech 631988) through whole gene synthesis.

Structure of H63 CAR sequence: CD8 signal peptide-H63-human CD8 hinge region-human CD8 transmembrane region-human 4-1BB costimulatory domain-CD3zeta signaling domain, and the nucleotide sequence is as set forth in SEQ ID NO: 19;

Structure of 21D4 CAR-2A-GFP sequence: CD8 signal peptide-21D4-human CD8 hinge region-human CD8 transmembrane region-human 4-1BB costimulatory domain-CD3zeta signaling domain-2A-green fluorescent protein, and the nucleotide sequence is as set forth in SEQ ID NO: 23;

Structure of 21D4 CAR-IRES-GFP sequence: CD8 signal peptide-21D4-human CD8 hinge region-human CD8 transmembrane region-human 4-1BB costimulatory domain-CD3zeta signaling domain-IRES-green fluorescent protein, and the nucleotide sequence is as set forth in SEQ ID NO: 24;

Structure of GPC3 CAR sequence: CD8 signal peptide-GPC3-human CD8 hinge region-human CD8 transmembrane region-human 4-1BB costimulatory domain-CD3zeta signaling domain, and the nucleotide sequence is as set forth in SEQ ID NO: 25;

Structure of GPC3 CAR-IRES-GFP sequence: CD8 signal peptide-GPC3-human CD8 hinge region-human CD8 transmembrane region-human 4-1BB costimulatory domain-CD3zeta signaling domain-IRES-green fluorescent protein, and the nucleotide sequence is as set forth in SEQ ID NO: 27;

Structure of CD19CD22 CAR sequence: CD8 signal peptide-CD19CD22-human CD8 hinge region-human CD8 transmembrane region-human 4-1BB costimulatory domain-CD3zeta signaling domain, and the nucleotide sequence is as set forth in SEQ ID NO: 28;

Structure of CD19CD22 CAR-IRES-GFP sequence: CD8 signal peptide-CD19CD22-human CD8 hinge region-human CD8 transmembrane region-human 4-1BB costimulatory domain-CD3zeta signaling domain-IRES-green fluorescent protein, and the nucleotide sequence is as set forth in SEQ ID NO: 30.

### Example 2. Preparation of Over-Expressed Lentiviruses

2.1 Formulation of Transfection Liquid

293T cells (ATCC CRL-3216) were plated in a T25 culture flask in DMEM culture solution containing 10% fetal bovine serum (Gibco, 10099-141) and 1% penicillin/streptomycin solution (Gibco, 15140-122), and cultured in an incubator to a cell density of up to 80-90% for transfection.

Reaction solution A and reaction solution B were formulated respectively as follows, mixed thoroughly and left to stand.

Reaction solution A was formulated following Table 1:

TABLE 1
Reaction system

| Reagents | Amount |
|---|---|
| Example 1 plasmid | 3.33 μg |
| psPAX2 plasmid (FENGHUI BIO TECH) | 2.50 μg |
| pMD2.G plasmid (FENGHUI BIO TECH) | 1.67 μg |
| Opti-MEM | 100 μl |

Reaction solution B was formulated following Table 2:

TABLE 2
Reaction system

| Reagents | Volume |
|---|---|
| PEIpro transfection reagent | 15 μl |
| Opti-MEM | 100 μl |

Reaction solution B was added dropwise into reaction solution A, mixed uniformly and let stand for 15 minutes.

293T cells were taken out of the incubator, onto the surface of which was uniformly added the mixed reaction solution dropwise, and then cultured in the incubator at 37° C. The culture solution was replaced after 16-24 h.

2.2 Concentration of Viruses

5 ml supernatant of the transfection reaction solution of plasmid 293T cells cultured in the T25 culture flask was pipetted into a centrifugal tube and centrifuged at 4800 rpm at 4° C. on a benchtop centrifuge for 10 minutes. The supernatant was pipetted into a new centrifugal tube, mixed well by Lenti-X Concentrator (TaKaRa, 631231) at a volume ratio of 1:3, and centrifuged at 1500×g at 4° C. on a benchtop centrifuge for 45 minutes. The supernatant was discarded, 250 μl PBS was added, mixed well, subpackaged, and stored at −80° C.

### Example 3. Viral Infection-Activated T Lymphocytes

Human T lymphocytes (Shanghai Sai Li Biological Technology Co. Ltd.) after being activated by CD3 antibodies and CD28 antibodies were cultured in 12-well cell culture plates. The culture solution was aspirated, and a viral solution containing different chimeric antigen receptor sequences was added and cultured in an incubator at 37° C. After 2 days of culture, the human T lymphocytes were blown up and transferred into T25 culture flasks, and 5 ml of T lymphocyte culture solution was added for suspension culture, so as to get CD19-targeted chimeric antigen receptor co-expressed TMEM59 protein chimeric antigen receptor T lymphocytes: H63 CAR-2A-TMEM59 CAR-T lymphocytes and 21D4-2A-TMEM59 CAR-T lymphocytes; CD19-targeted TMEM59 modified chimeric antigen receptor T lymphocytes, as well as control CAR-T lymphocytes: H63 CAR-T lymphocytes and 21D4-2A-GFP CAR-T lymphocytes.

### Example 4. Memory Testing of CD19-Targeted Chimeric Antigen Receptor Co-Expressed TMEM59 Protein Chimeric Antigen Receptor T Lymphocytes

H63 CAR-T lymphocytes, H63 CAR-2A-TMEM59 CAR-T lymphocytes, 21D4-2A-GFP CAR-T lymphocytes and 21D4-2A-TMEM59 CAR-T lymphocytes obtained from Example 3 were adequately suspended respectively. After counting, a total amount of 1×10⁶ cells were transferred into flow-through tubes. The cells were washed with phosphate buffer salt solution twice and centrifuged at a centrifugal parameter of 400×g at room temperature for 5 minutes. The supernatant was discarded, remaining 100 μl of liquid, into which was added 1 μg of biotin-protein L (ACROBIOSYSTEMS, RPL-P814R) at 1 μl per tube, and they were incubated at 4° C. in the dark conditions for 30 minutes. Phosphate buffer salt solution was used to wash twice, remaining 100 μl of liquid, into which was added 1 μl PE-streptomycin (BD, 554061), 5 μl CD45 RA antibody (Biolegend, 304122) and 5 μl CD 62L antibody (BD, 565535), and they were incubated at 4° C. in the dark conditions for 30 minutes. After washing twice with the phosphate buffer salt solution, 400 μl of phosphate buffer salt solution was added for adequate suspension. A flow cytometry testing was performed by a flow cytometer. The light chain portion of the single-chain variable region of the CAR protein was labelled with protein L, representing positive chimeric antigen receptor T lymphocytes. CD45RA and CD62L were the memory labels on the surface of T lymphocytes, and the increased expression level of CD45RA and CD62L indicated the enhanced memory of T lymphocytes.

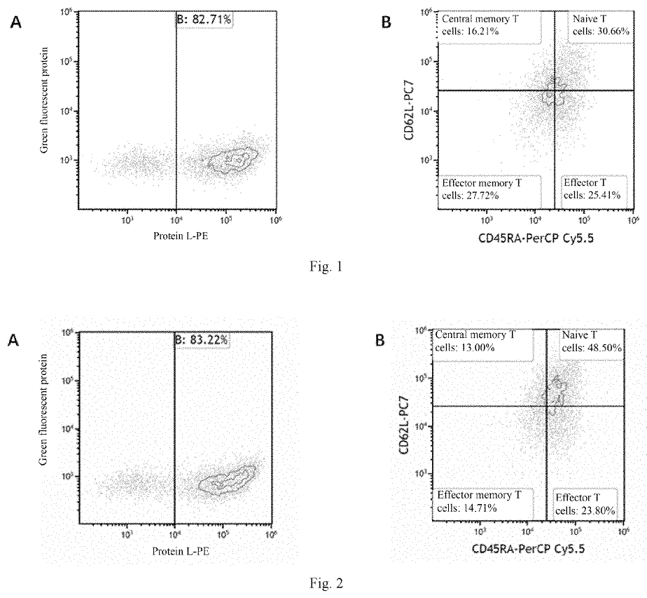

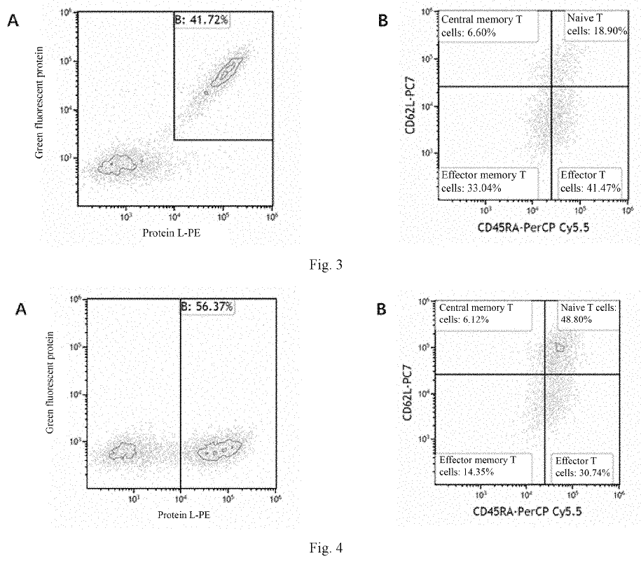

The results were shown in FIGS. 1-4 and Table 3 below. Table 3 showed the number of memory cells. Compared to H63 CAR-T lymphocytes (FIG. 1), the proportion of memory cells in H63 CAR-2A-TMEM59 T lymphocytes (FIG. 2) raised from 30.66% (FIG. 1B) up to 48.50% (FIG. 2B). Similarly, compared to 21D4 CAR-2A-GFP T lymphocytes (FIG. 3), the proportion of memory cells in 21D4 CAR-2A-TMEM59 T lymphocytes (FIG. 4) raised from 18.90% (FIG. 3B) up to 48.80% (FIG. 4B). It was indicated that the additional expression of TMEM59 protein by CD19 CAR-T lymphocytes could increase the number of memory cells in CD19 CAR-T lymphocytes.

TABLE 3
Number of lymphocytes

| | CAR positive cells | Naive T |
|---|---|---|
| Samples | % (protein L-positive) | lymphocytes % |
| H63 CAR-T lymphocytes | 82.71 | 30.66 |
| H63 CAR-2A-TMEM59-T | 83.22 | 48.50 |
| lymphocytes | | |
| 21D4 CAR-2A-GFP-T | 41.72 | 18.90 |
| lymphocytes | | |
| 21D4 CAR-2A-TMEM59-T | 56.37 | 48.80 |
| lymphocytes | | |

### Example 5. Proliferative Ability Testing of CD19-Targeted Chimeric Antigen Receptor Co-Expressed TMEM59 Protein Chimeric Antigen Receptor T Lymphocytes

CAR-T cells obtained from Example 3 were cultured in an incubator at 37° C. After 2 days of culture, the cells were blown up and transferred into T25 culture flasks, into which 5 ml of T lymphocyte culture solution was added for suspension culture. On day 4 of culture, 5 ml of T lymphocyte culture solution was supplemented. The cells were blown uniformly with a 1 ml gun and suspended adequately. 20 μl of cell suspension was immediately taken and injected into the sampling holes of the counting plate, and counted by a Cellometer Auto 2000 cell counter. After counting, there was a total of 10 ml cell liquid. 5 ml of cell suspension was discarded, and 5 ml of fresh culture solution was supplemented into the culture solution. The above steps were repeated after 24 hours, and the cells were counted, until day 8.

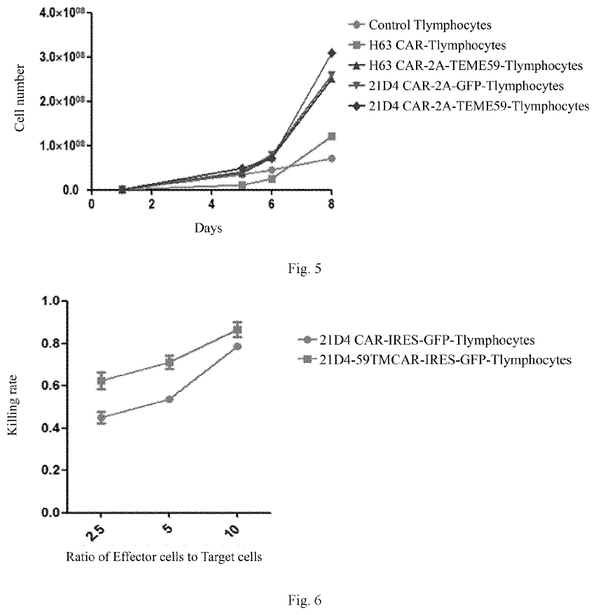

The proliferative number of cells was shown in FIG. 5. Compared to H63 CAR-T lymphocyte, the cell number of H63 CAR-2A-TMEM59-T lymphocytes increased. Similarly, compared to 21D4 CAR-2A-GFP-T lymphocytes, the cell number of 21D4 CAR-2A-TMEM59-T lymphocytes increased. It was indicated that under cultivation conditions, TMEM59 was helpful to improve the proliferative ability of CART lymphocytes.

### Example 6. Memory Testing of CD19-Targeted TMEM59 Modified Chimeric Antigen Receptor T Lymphocytes

The memory of CD19-targeted TMEM59 modified chimeric antigen receptor T lymphocytes obtained from Example 3 was tested by a flow cytometer according to the method of Example 4.

The results showed that, compared to that in 21D4 CAR-IRES-GFP-T lymphocytes, the ratio of the number of memory cells in 21D4 59TM CAR-IRES-GFP-T lymphocytes increased, indicating that the TMEM59 modified CD19 CAR-T lymphocytes were able to enhance the number of memory cells in CD19 CAR-T lymphocytes.

### Example 7. Killing Ability Testing of CD19-Targeted TMEM59 Modified Chimeric Antigen Receptor T Lymphocytes Against Target Cells NALM-6 (Luciferase Method)

Lentivirus-infected T lymphocytes were cultured, and plated in 96-well round-bottom plates together with NALM-6 cells (Imanis LIFE SCIENCE, CL151) at ratios of effector cells (T lymphocytes): target cells (NALM-6 cells) of 10:1, 5:1, 2.5:1, wherein the amount of NALM-6 cells was 1×10⁴/well. The culture system was RPMI-1640 culture solution containing 10% fetal bovine serum, with a total of 200 ul cell suspension per well.

The samples were grouped as shown in Table 4 below, with 3 replications for each group:

TABLE 4
Ratio of effector cells to target cells

| 10:1 | 5:1 | 2.5:1 | 0 |
|---|---|---|---|
| 21D4 | 21D4 | 21D4 | NALM-6 |
| CAR-IRES- | CAR-IRES- | CAR-IRES- | cells |
| GFP-T | GFP-T | GFP-T | |
| lymphocytes + | lymphocytes + | lymphocytes + | |
| NALM-6 | NALM-6 | NALM-6 | |
| cells | cells | cells | |
| 21D4-59TM | 21D4-59TM | 21D4-59TM | NALM-6 |
| CAR-IRES- | CAR-IRES- | CAR-IRES- | cells |
| GFP-T | GFP-T | GFP-T | |
| lymphocytes + | lymphocytes + | lymphocytes + | |
| NALM-6 | NALM-6 | NALM-6 | |
| cells | cells | cells | |

96-well culture plates were cultured in an incubator at 37° C. for 4 hours. The testing was conducted following the instruction for the luciferase kit (promega, E2920). The 96-well culture plates that had been cultured for 4 hours were taken out, 175 μl of cell mixture was aspirated and added into the wells of clear bottom black plate 96-well culture plates, into which luciferase substrate was added at 25 μl/well, and incubated in dark at room temperature for 10 minutes, and then detected by a microplate reader.

The results were shown in FIG. 6. In this Example, the number of surviving NALM-6 cells was reflected by the activity of luciferase expressed by NALM-6 cells. Both CD19 (21D4)-targeted TMEM59 modified chimeric antigen receptor T lymphocytes (21D4-59TM CAR-IRES-GFP) and CD19 (21D4)-targeted chimeric antigen receptor T lymphocytes (21D4 CAR-IRES-GFP) were able to lyse NALM-6 cells, and the lysis of NALM-6 cells by 21D4-59TM CAR-IRES-GFP-T lymphocytes was slightly higher than that by 21D4 CAR-2A-GFP-T lymphocytes.

### Example 8. Proliferative Ability Testing of GPC3-Targeted Chimeric Antigen Receptor Co-Expressed TMEM59 Protein Chimeric Antigen Receptor T Lymphocytes

The proliferative ability of GPC3-targeted chimeric antigen receptor co-expressed TMEM59 protein chimeric antigen receptor T lymphocytes obtained from Example 3 was tested according to the method of Example 5.

The results showed that, the cell number of GPC3-targeted chimeric antigen receptor co-expressed TMEM59 protein chimeric antigen receptor T lymphocytes (GPC3 CAR-IRES-TEME59-T lymphocytes) was increased compared to that of GPC3 CAR-T lymphocytes and GPC3 CAR-IRES-GFP-T lymphocytes.

### Example 9. Memory Testing of GPC3-Targeted TMEM59 Modified Chimeric Antigen Receptor T Lymphocytes

The memory of GPC3-targeted TMEM59 modified chimeric antigen receptor T lymphocytes was tested by a flow cytometer according to the method of Example 4.

The results showed that, compared to that in GPC3 CAR-IRES-GFP T lymphocytes, the ratio of the number of memory cells in GPC3-59TM CAR-IRES-GFP-T lymphocytes increased, indicating that the TMEM59 modified GPC3 CAR-T lymphocytes were able to enhance the number of memory cells in GPC3 CAR-T lymphocytes.

### Example 10. Killing Ability Testing of GPC3-Targeted TMEM59 Modified Chimeric Antigen Receptor T Lymphocytes Against Target Cells Huh7 (Luciferase Method)

The killing ability of the GPC3-targeted TMEM59 modified chimeric antigen receptor T lymphocytes obtained from Example 3 against target cells Huh7 was tested according to the method of Example 7. The samples were grouped as shown in Table 5 below, with 3 replications for each group:

TABLE 5
Ratio of effector cells to target cells

| 10:1 | 5:1 | 2.5:1 | 0 |
|---|---|---|---|
| GPC3 | GPC3 | GPC3 | Huh7 |
| CAR-IRES- | CAR-IRES- | CAR-IRES- | cells |
| GFP-T | GFP-T | GFP-T | |
| lymphocytes + | lymphocytes + | lymphocytes + | |
| Huh7 cells | Huh7 cells | Huh7 cells | |
| GPC3-59TM | GPC3-59TM | GPC3-59TM | Huh7 |
| CAR-IRES- | CAR-IRES- | CAR-IRES- | cells |
| GFP-T | GFP-T | GFP-T | |
| lymphocytes + | lymphocytes + | lymphocytes + | |
| Huh7 cells | Huh7 cells | Huh7 cells | |

The results showed that, both GPC3-targeted TMEM59 modified chimeric antigen receptor T lymphocytes (GPC3-59TM CAR-IRES-GFP) and GPC-targeted chimeric antigen receptor T lymphocytes (GPC3 CAR-2A-GFP) were able to lyse Huh7 cells, and the lysis of Huh7 cells by GPC3-59TM CAR-IRES-GFP-T lymphocytes was higher than that by GPC3 CAR-IRES-GFP-T lymphocytes.

### Example 11. Proliferative Ability Testing of CD19 CD22-Targeted Chimeric Antigen Receptor Co-Expressed TMEM59 Protein Chimeric Antigen Receptor T Lymphocytes

The proliferative ability of CD19 CD22-targeted chimeric antigen receptor co-expressed TMEM59 protein chimeric antigen receptor T lymphocytes obtained from Example 3 was tested according to the method of Example 5.

The results showed that, the cell number of CD19 CD22-targeted chimeric antigen receptor co-expressed TMEM59 protein chimeric antigen receptor T lymphocytes (CD19CD22 CAR-IRES-TEME59-T lymphocytes) was increased compared to that of CD19CD22 CAR-T lymphocytes and CD19CD21 CAR-IRES-GFP-T lymphocytes.

### Example 12. Memory Testing of CD19 CD22-Targeted TMEM59 Modified Chimeric Antigen Receptor T Lymphocytes

The memory of CD19 CD22-targeted TMEM59 modified chimeric antigen receptor T lymphocytes obtained from Example 3 was tested by a flow cytometer according to the method of Example 4.

The results showed that, compared to that in CD19CD22 CAR-IRES-GFP T lymphocytes, the ratio of the number of memory cells in CD19CD22-59TM CAR-IRES-GFP-T lymphocytes increased, indicating that the TMEM59 modified CD19CD22 CAR-T lymphocytes were able to enhance the number of memory cells in CD19CD22 CAR-T lymphocytes.

### Example 13. Killing Ability Testing of CD19 CD22-Targeted TMEM59 Modified Chimeric Antigen Receptor T Lymphocytes Against Target Cells NALM-6 (Luciferase Method)

The killing ability of the CD19 CD22-targeted TMEM59 modified chimeric antigen receptor T lymphocytes obtained from Example 3 against target cells NALM-6 was tested according to the method of Example 7. The samples were grouped as shown in Table 6 below, with 3 replications for each group:

TABLE 6
Ratio of effector cells to target cells

| 10:1 | 5:1 | 2.5:1 | 0 |
|---|---|---|---|
| CD19CD22 | CD19CD22 | CD19CD22 | NALM-6 |
| CAR-IRES- | CAR-IRES- | CAR-IRES- | cells |
| GFP-T | GFP-T | GFP-T | |
| lymphocytes + | lymphocytes + | lymphocytes + | |
| NALM-6 | NALM-6 | NALM-6 | |
| cells | cells | cells | |
| CD19CD22- | CD19CD22- | CD19CD22- | NALM-6 |
| 59TM | 59TM | 59TM | cells |
| CAR-IRES- | CAR-IRES- | CAR-IRES- | |
| GFP-T | GFP-T | GFP-T | |
| lymphocytes + | lymphocytes + | lymphocytes + | |
| NALM-6 | NALM-6 | NALM-6 | |
| cells | cells + | cells + | |
| | NALM-6 | NALM-6 | |
| | cells | cells | |

The results showed that, both CD19 CD22-targeted TMEM59 modified chimeric antigen receptor T lymphocytes (CD19 CD22-59TM CAR-IRES-GFP) and CD19 CD22-targeted chimeric antigen receptor T lymphocytes (CD19 CD22 CAR-2A-GFP) were able to lyse NALM-6 cells, and the lysis of NALM-6 cells by CD19 CD22-59TM CAR-IRES-GFP-T lymphocytes was higher than that by CD19 CD22 CAR-IRES-GFP-T lymphocytes.

### Example 14. Inhibition of Tumor Growth in Mice by Chimeric Antigen Receptor

Co-Expressed TMEM59 and TMEM59 Modified Chimeric Antigen Receptor Chimeric Antigen Receptor T Lymphocytes

14.1 Establishment of Tumor-Bearing Mouse Models

Self-constructed target tumor cells expressing luciferase were inoculated into NSG mice for subcutaneous injection into tumors. 2×10⁵ of the above target cells were resuspended in 100 μl of phosphate buffer salt solution for subcutaneous injection.

Alternatively, self-constructed target tumor cells expressing luciferase, Nalm-6 cells, were inoculated into NSG mice for intravenous injection into tumors. 2.5×10⁵ of the above target cells were resuspended in 100 μl of phosphate buffer salt solution for intravenous injection into mouse tail.

14.2 In Vivo Imaging

3 days after tumor formation, Luciferin, a luciferase substrate, was intraperitoneally injected into mice at an injection amount of 3 mg per mouse. After anesthetization, the mice were placed in a small animal in vivo imager for imaging. 7 days after the injection of Nalm cells, T lymphocytes expressing CD19CD22-59TM CAR (whose nucleotide sequence was shown in SEQ ID NO: 31) were washed with PBS and resuspended in serum-free culture solution, and the cell density was adjusted to 2×10⁶/ml. Each mouse was injected with 2×10⁶ cells at the tail vein, with a total of 200 μl. Mice without treatment were set as a control group. After the completion of the experiment, in vivo imaging was performed periodically to collect the effect of tumor elimination. Biofluorescence assays were performed every 3-7 days, and a recurrence model challenge was performed by injecting the CAR-T dosing group with 2.5×10⁵ NALM6 tumor cells on day 60.

14.3 Measurement of Tumor Size

After injection of cells, the weight was measured twice a week, with the day of cell injection as day 0.

14.4 Measurement of Mouse Weight

After injection of cells, the tumor size was measured twice a week, with the day of cell injection as day 0. The tumor size was calculated as below: tumor size (mm³)=0.5×long diameter of tumor×short diameter of tumor².

14.5 Measurement of Tumor Weight

At the end of the test, tumor-bearing mice were euthanatized, and the tumor was peeled and weighed.

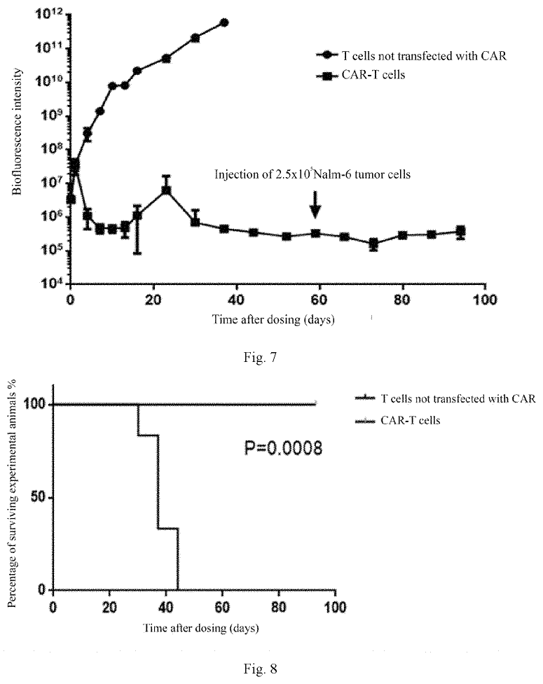

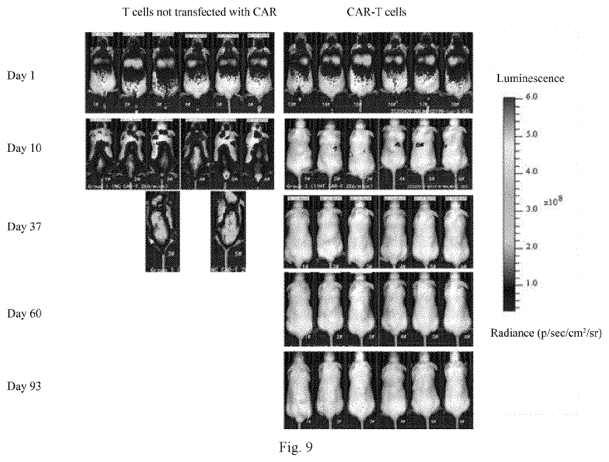

The results showed that, compared to mice in the control group, mice injected with CAR-T cells showed reduced tumor size, reduced tumor weight and slowed weight gain trend. FIG. 7 showed the biofluorescence intensity, reflecting the number of tumor cells. The results showed that, compared to the group of T cells not transfected with CAR and with increasing biofluorescence intensity, the number of tumor cells in the mice in the group of T cells transfected with CD19CD22-59TM CAR was significantly reduced, and the fluorescence intensity was approximately between 10⁵ and 10⁶ and did not increase even after reinjection of tumor cells on day 60. FIG. 8 showed the percentage of surviving experimental animals in the experimental and control groups. The survival rate of mice in the group of T cells not transfected with CAR, served as the control, decreased approximately from day 30 to 0 approximately on day 45. However, the survival rate of mice in the group of T cells transfected with CD19CD22-59TM CAR still remained at 100% after 90 days. FIG. 9 showed the in vivo imaging results of mice. For mice in the group of T cells not transfected with CAR, the light intensity did not decrease on day 10 and day 37, and the number of mice decreased on day 37 because of death. While for mice in the group of T cells transfected with CD19CD22-59TM CAR, the light intensity significantly decreased on day 10, and no light intensity could be detected after day 37, indicating that the tumor had been eliminated.

During the treatment, no animals died, and no obvious drug toxicity was shown, indicating good tolerance.

The foregoing detailed description is provided by way of explanation and examples, and is not intended to limit the scope of the appended claims. Various changes of the embodiments currently enumerated in the present application will be apparent to those of ordinary skills in the art, and are reserved within the scope of the appended claims and their equivalents.

1. An immune cell, comprising a TMEM59 protein and/or a functional fragment thereof located on the cell membrane, wherein said functional fragment comprises at least a transmembrane region and a hinge region of said TMEM59 protein.

2. The immune cell according to claim 1, which is modified to comprise said TMEM59 protein and/or the functional fragment thereof on the cell membrane.

3. The immune cell according to claim 1, wherein said TMEM59 protein and/or the functional fragment thereof is a human TMEM59 protein and/or a functional fragment thereof.

4. The immune cell according to claim 1, wherein said transmembrane region of the TMEM59 protein comprises an amino acid sequence as set forth in SEQ ID NO. 1, wherein said hinge region of the TMEM59 protein comprises an amino acid sequence as set forth in SEQ ID NO. 2, and/or wherein said TMEM59 protein and/or the functional fragment thereof comprises an amino acid sequence as set forth in SEQ ID NO. 3.

5. (canceled)

6. (canceled)

7. The immune cell according to claim 1, wherein said immune cell comprises a chimeric antigen receptor (CAR) and/or a coding element thereof.

8. The immune cell according to claim 7, wherein said CAR comprises a transmembrane region, a hinge region, and/or an intracellular domain, wherein said intracellular domain of the CAR comprises a signaling domain, a costimulatory domain and/or a targeting moiety.

9. The immune cell according to claim 8, wherein said transmembrane region of the CAR is selected from: a transmembrane region of said TMEM59 protein and a transmembrane region of CD8; wherein said hinge region of the CAR is selected from: a hinge region of said TMEM59 protein and a hinge region of CD8, wherein said signaling domain comprises a signaling domain of CD3zeta, and/or wherein said costimulatory domain comprises a costimulatory domain of 4-1BB.

10. The immune cell according to claim 8, wherein said transmembrane region of the CAR comprises an amino acid sequence as set forth in any one of SEQ ID NOs. 1 and 37, and/or wherein said hinge region of the CAR comprises an amino acid sequence as set forth in any one of SEQ ID NOs. 2 and 38.

11. (canceled)

12. (canceled)

13. (canceled)

14. (canceled)

15. (canceled)

16. (canceled)

17. (canceled)

18. (canceled)

19. (canceled)

20. The immune cell according to claim 8, wherein said targeting moiety specifically binds to and/or recognizes a target selected from a group consisting of: CD19, CD22 and GPC3.

21. The immune cell according to claim 9, comprising said CAR as well as said TMEM59 protein and/or the functional fragment thereof, and wherein said CAR does not comprise said TMEM59 protein and/or the functional fragment thereof.

22. The immune cell according to claim 21, wherein said CAR comprises said targeting moiety, said hinge region of CD8, said transmembrane region of CD8, said signaling domain of CD3zeta and said costimulatory domain of 4-1BB, wherein said TMEM59 protein and/or the functional fragment thereof comprises said transmembrane region and said hinge region of the TMEM59 protein.

23. The immune cell according to claim 21, wherein said CAR comprises an amino acid sequence as set forth in any one of SEQ ID NOs. 49, 51, 55 and 56.

24. The immune cell according to claim 9, comprising said CAR as well as said TMEM59 protein and/or the functional fragment thereof, and wherein said CAR comprises said TMEM59 protein and/or the functional fragment thereof.

25. The immune cell according to claim 24, wherein said CAR comprises said targeting moiety, said hinge region of the TMEM59 protein, said transmembrane region of the TMEM59 protein, said signaling domain of CD3zeta and said costimulatory domain of 4-1BB, wherein said TMEM59 protein and/or the functional fragment thereof comprises said transmembrane region and said hinge region of the TMEM59 protein.

26. The immune cell according to claim 24, wherein said CAR comprises an amino acid sequence as set forth in any one of SEQ ID NOs. 43, 57 and 44.

27. The immune cell according to claim 1, wherein said immune cell comprises a T lymphocyte.

28. A pharmaceutical composition, comprising the immune cell according to claim 1 and optionally a pharmaceutically acceptable carrier.

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. A method for inhibiting the proliferation of tumor cells and/or killing tumor cells, comprising: contacting the immune cell of claim 1 with said tumor cells.

41. (canceled)

42. The method according to claim 40, wherein said tumor comprises a solid tumor and/or a non-solid tumor.

43. The method according to claim 40, wherein said tumor comprises B lymphocytic leukemia and/or liver cancer.
